(19) United States
(12) Patent Application Publication
(10) Pub. No.: US 2026/0132385 A1
(43) Pub. Date: May 14, 2026

(54) NOVEL ANION-EXCHANGE CHROMATOGRAPHY METHODS FOR SEPARATION OF EMPTY FROM FULL RECOMBINANT ADENO-ASSOCIATED VIRUS PARTICLES

(71) Applicant: Ultragenyx Pharmaceutical Inc., Novato, CA (US)

(72) Inventors: Wenjun Di, Woburn, MA (US); Dennis Perry Chen, Boston, MA (US); Kyle Koczera, Woburn, MA (US); Amy Medeiros, Melrose, MA (US); Ying Cai, Lexington, MA (US); Chao Huang, Belmont, MA (US); James C. Warren, Hollis, NH (US)

(21) Appl. No.: 19/102,000

(22) PCT Filed: Aug. 10, 2023
(86) PCT No.: PCT/US2023/071986

§ 371 Date: Feb. 7, 2025

Related U.S. Application Data
(60) Provisional application No. 63/397,668, filed on Aug. 12, 2022.
(60) Provisional application No. 63/507,443, filed on Jun. 9, 2023.

(51) Int. Cl. C12N 7/00; B01D 15/18; B01D 15/20; B01D 15/36; B01D 15/42; B01J 41/20

(52) U.S. Cl. CPC C12N 7/00; B01D 15/1864; B01D 15/203; B01D 15/363; B01D 15/424; B01J 41/20; C12N 2750/14151

(57) ABSTRACT

Provided are methods of separating full capsid particles and empty capsid particles in a viral capsid preparation by overloading viral capsid preparations onto an anion exchange medium integrated with weak partitioning chromatography and multiple column chromatography (AEX-WPC-MCC). Also provided are viral capsid loading ranges for overloading AEX medium facilitating separating full and empty capsid particles.

### CROSS-REFERENCE TO RELATED APPLICATIONS

This application claims the benefit of and priority to U.S. Provisional Patent Application Nos. 63/397,668, filed on Aug. 12, 2022, and 63/507,443, filed on Jun. 9, 2023, the disclosures of each which are incorporated by reference herein in its entirety for all purposes.

### BACKGROUND

Recombinant adeno-associated viruses (rAAVs) engineered to carry a heterologous nucleic acid of interest (e.g., a gene encoding a therapeutic protein, an antisense nucleic acid molecule, a ribozyme, a miRNA, an siRNA, a nucleic acid encoding a CRISPR/Cas system, or the like) are increasingly being explored as therapeutic agents for various diseases. These rAAVs are engineered by deleting, in whole or in part, the internal portion of the AAV genome and inserting the heterologous nucleic acid of interest between the inverted terminal repeats (ITRs). The ITRs remain functional in such vectors allowing replication and packaging of the AAV particle containing the nucleic acid cargo enclosed within the AAV capsid. Typically, the heterologous nucleic acid is operably linked to regulatory sequences (e.g., promoter and/or enhancer sequences) capable of driving expression of the cargo in a patient's target cells.

Large scale manufacturing of these rAAVs may suffer from inefficiencies in the packaging of the nucleic acid cargo into viral capsids leading to rAAV preparations that include a mixture of “full” AAV particles (i.e., particles that include the nucleic acid of interest) and “empty” AAV particles (i.e., AAV particles that lack in whole or in part the nucleic acid cargo). The presence of empty AAV particles in a gene therapy product may necessitate increasing the overall dose of the rAAV preparation (e.g., in the form of a pharmaceutical composition comprising such rAAV preparation) administered to a patient to achieve therapeutic efficacy and may promote or exacerbate an immune response (e.g., development of neutralizing antibodies or T-cell activation) upon administration of the rAAV preparation to a patient.

In rAAV downstream purification processes, anion-exchange (AEX) chromatography has been utilized as a major scalable method for empty and full rAAV separation. Although multiple AEX factors, including AEX stationary phase, AEX mobile phase, and AEX elution mode, have been evaluated to improve full rAAV percentage, the challenge of empty/full separation still remains for rAAV viral products, which is thought to stem predominantly from the minimal charge difference between empty and full rAAVs and the charge heterogeneity of both rAAV species. For example, when using salt linear gradient elution AEX chromatography, the empty rAAV peak elutes just a few milliseconds per centimeter (mS/cm) from the full rAAV peak, and the co-elution of empty and full rAAVs in the AEX pool seems to be inevitable.

### SUMMARY

There is a need in the art for new purification methods that can be used to separate full AAV particles from empty AAV particles in rAAV preparations. The present disclosure addresses this need with a solution based in part on the insight that the separation of full capsid particles and empty capsid particles in viral capsid preparations (e.g., rAAV preparations) using anion-exchange (AEX) chromatography can be improved by integrating weak partitioning chromatography and multiple chromatography techniques into the AEX process, said techniques comprising overloading the AEX medium with a rAAV preparation (i.e., a loading material/rAAV capsid preparation), wherein the rAAV loading material comprises a specific range of viral particles. In some embodiments, the present disclosure provides methods of integrating weak partitioning chromatography (WPC) and multiple-column chromatography (MCC) into AEX methods, providing improved empty/full rAAV separation, where the empty rAAVs preferentially flow through the AEX column during or as a result of column overloading. In particular embodiments, the present disclosure provides an AEX-WPC-MCC rAAV purification method that results in the enhancement of both viral genome recovery and full rAAV % of an AEX pool, and reduces the AEX column volume and buffer usage, contributing to improved efficiency and cost reduction of downstream rAAV processing.

In some embodiments, the present disclosure provides a method of separating empty capsid particles in a viral capsid preparation comprising empty and full capsid particles, the method comprising the steps of a) applying an equilibrating solution to at least one anion exchange (AEX) medium; and b) applying a volume of the viral capsid preparation to the at least one AEX medium, and passing the volume of the viral capsid preparation through the at least one AEX medium to generate a flowthrough comprising empty capsid particles, wherein the volume of the viral capsid preparation comprises between 1×10¹⁴ to 5.5×10¹⁵ vp/ml of the AEX medium.

In some embodiments, the present disclosure provides a method of separating empty capsid particles in a viral capsid preparation comprising empty and full capsid particles, the method comprising the steps of: a) applying an equilibrating solution to at least one anion exchange (AEX) medium; and b) passing a volume of the viral capsid preparation through the at least one AEX medium to generate a flowthrough comprising empty capsid particles, wherein the volume of the viral capsid preparation comprises between 1×10¹⁴ to 5.5×10¹⁵ vp/ml of the AEX medium.

In some embodiments, the present disclosure provides a method of separating full capsid particles and empty capsid particles in a viral capsid preparation comprising empty and full capsid particles, the method comprising the steps of: a) applying an equilibrating solution to at least one anion exchange (AEX) medium; b) applying a volume of the viral capsid preparation to the at least one AEX medium and passing the volume of the viral capsid preparation through the at least one AEX medium to generate a flowthrough comprising empty capsid particles; and c) applying an elution solution comprising a salt to the at least one AEX medium and passing the elution solution through the at least one AEX medium to generate an eluent comprising full capsid particles, wherein the volume of the viral capsid preparation comprises between 1×10¹⁴ to 5.5×10¹⁵ vp/ml of the AEX medium.

In some embodiments, the present disclosure provides a method of separating full capsid particles and empty capsid particles in a viral capsid preparation comprising empty and full capsid particles, the method comprising the steps of: a) applying an equilibrating solution to at least one anion exchange (AEX) medium; b) passing a volume of the viral capsid preparation through the at least one AEX medium to generate a flowthrough comprising empty capsid particles; and c) passing an elution solution comprising a salt through the at least one AEX medium to generate an eluent comprising full capsid particles, wherein the volume of the viral capsid preparation comprises between 1×10¹⁴ to 5.5×10¹⁵ vp/ml of the AEX medium.

The present disclosure also provides a method of increasing the percentage of full capsid particles in a viral capsid preparation comprising empty and full capsid particles, the method comprising the steps of: a) applying an equilibrating solution to at least one anion exchange (AEX) medium; b) applying a volume of the viral capsid preparation to the at least one AEX medium, and passing the volume of the viral capsid preparation through the at least one AEX medium to generate a flowthrough; c) applying an elution solution comprising a salt to the at least one AEX medium and passing the elution solution through the at least one AEX medium to generate an eluent; d) collecting the eluent from step c), wherein a volume of the eluent of step d) comprises full capsid particles at a percentage that is higher than the percentage of full capsid particles in an equivalent volume of the viral capsid preparation, and wherein the volume of the viral capsid preparation comprises between 1×10¹⁴ to 5.5×10¹⁵ vp/ml of the AEX medium.

The present disclosure also provides a method of increasing the percentage of full capsid particles in a viral capsid preparation comprising empty and full capsid particles, the method comprising the steps of: a) applying an equilibrating solution to at least one anion exchange (AEX) medium; b) passing a volume of the viral capsid preparation through the at least one AEX medium to generate a flowthrough; c) passing an elution solution comprising a salt through the at least one AEX medium to generate an eluent; and d) collecting the eluent from step c), wherein a volume of the eluent of step d) comprises full capsid particles at a percentage that is higher than the percentage of full capsid particles in an equivalent volume of the viral capsid preparation, and wherein the volume of the viral capsid preparation comprises between 1×10¹⁴ to 5.5×10¹⁵ vp/ml of the AEX medium.

The present disclosure also provides an elution method comprising the steps of: a) contacting at least one anion exchange (AEX) medium with a volume of a viral capsid preparation comprising full capsid particles and empty capsid particles and passing the volume of viral capsid preparation through the AEX medium to generate a flowthrough comprising empty capsid particles; and b) contacting the at least one AEX medium with an elution solution comprising a salt and passing the elution solution through the at least one AEX medium to generate an eluent comprising full capsid particles, wherein the volume of the viral capsid preparation comprises between 1×10¹⁴ to 5.5×10¹⁵ vp/ml of the AEX medium.

The present disclosure also provides an elution method comprising the steps of a) passing a volume of viral capsid preparation through at least one anion exchange (AEX) medium to generate a flowthrough comprising empty capsid particles; and b) passing an elution solution comprising a salt through the at least one AEX medium to generate an eluent comprising full capsid particles, wherein the volume of the viral capsid preparation comprises between 1×10¹⁴ to 5.5×10¹⁵ vp/ml of the AEX medium.

### BRIEF DESCRIPTION OF THE DRAWINGS

FIG. 1A and FIG. 1B depict gradient elution profiles of normal batch loading AEX chromatography. FIG. 1A depicts chromatograms showing a gradient elution profile of normal batch loading of rAAV load material comprising 4% full rAAV, using 1 ml CIMmultus®-QA Column. FIG. 1B depicts chromatograms showing gradient elution profile of normal batch loading of rAAV loading material/capsid preparations comprising 4% full rAAV, using 0.1 ml CIMac®-QA Column. The UV280 (mAU), UV254 (mAU), and conductivity (mS/cm) traces in AEX salt linear gradient elution phase are shown by a thin solid line, thick solid line, and dashed line, respectively. With conductivity increase, empty rAAV peak (denoted as “E”), full rAAV peak (denoted as “F”), and third peak (denoted as “T”) were sequentially resolved in salt linear gradient.

FIGS. 2A-2C depict results from AEX dynamic binding capacity (DBC) run experiments using 11% full rAAV load material on 1 ml CIMmultus®-QA column. FIG. 2A breakthrough curves in AEX DBC run using 11% Full rAAV Load Material on 1 ml CIMmultus®-QA Column. The UV280 (mAU) and UV254 (mAU) traces in AEX loading phase are shown by a thin solid line and thick solid line, respectively. The viral particle concentration (vp/ml) and viral genome concentration (vg/ml) of the selected flowthrough fractions are shown in open bar graph and hatched bar graph, respectively. FIG. 2B depicts chromatograms showing gradient elution profile of AEX DBC run using 11% Full rAAV load material on 1 ml CIMmultus®-QA Column. FIG. 2C depicts chromatograms showing Gradient Elution Profile of AEX Normal Batch Loading Run using 11% Full rAAV Load Material on 1 ml CIMmultus®-QA Column. The UV280 (mAU), UV254 (mAU), and conductivity (mS/cm) traces in AEX salt linear gradient elution phase are shown by a thin solid line, a thick solid line, and dashed line, respectively. With conductivity increase, empty rAAV peak (denoted as “E”), full rAAV peak (denoted as “F”), and third peak (denoted as “T”) were sequentially resolved in salt linear gradient.

FIGS. 3A-3F are schematics depicting the operation scheme of multi-column chromatography for AEX-WPC-MCC Run. Taken together, all six operation schematics (FIGS. 3A-3F) depict sequential operation of one loop of 3-column MCC operation. Each column is indicated as column 1, 2 or 3. The equilibration phase (Eq), loading phase (L), gradient elution phase (G), cleaning phase (C) and idle phase (I), for each column as indicated, are done in a sequential manner. FIG. 3A depicts E phase for column 1 and I phase for both column 2 and column 3. FIG. 3B depicts loading phase for column 1, sequential Eq phases for column 2 and column 3; FIG. 3C depicts gradient elution phase plus cleaning phase plus Eq phase for column 1, L phase for column 2, and I phase for column 3. FIG. 3D depicts C phase for column 1, plus C phase for column 2, and L phase for column 3. FIG. 3E depicts L phase for column 1, I phase for column 2, and G phase plus C phase for column 3; FIG. 3F depicts G phase plus C phase for column 1, I phase for both column 2 and column 3. In all diagrams, circled “B” with black arrow represents buffer pump delivers buffers to the columns, while circled “S” with black arrow represents sample pump delivers load material to the columns.

FIGS. 4A-4C depicts graphs showing breakthrough curves from AEX dynamic binding capacity (DBC) runs of rAAV load material on 0.1 ml CIMac®-QA column showing loading range (low and high end loading) of viral load material. FIG. 4A depicts the breakthrough curves in AEX DBC run using 11% full rAAV load material on 0.1 ml CIMac®-QA column. FIG. 4B depicts breakthrough curves in AEX dynamic binding Run using 4% full rAAV load material on 0.1 ml CIMac®-QA column. The breakthrough percentage of viral particle concentrations (vp/ml) and viral genome concentrations (vg/ml) are shown in open squares connected by a solid line and open circles connected a solid line, respectively. The HCDNA concentrations (ng/1+12 vg) are shown in open triangles. In FIG. 4B, the encircled letter “L” indicates the loading condition of low-end loading AEX-WPC-MCC run, while the encircled letter “H” indicates the loading condition of high-end loading AEX-WPC-MCC-run. FIG. 4C depicts the relationship of % breakthrough in terms of viral particle (squares) and viral genome (circles), with respect to column loading, to determine the operation space for the AEX-WPC-MCC overloading run.

FIGS. 5A-5F depicts the loading profiles, gradient elution profiles and full viral AAV capsid recovery of low end and high end viral capsid preparation/loading material on AEX-WPC-MCC runs. FIG. 5A shows chromatograms depicting loading profile of low-end loading AEX-WPC-MCC run using 11% full rAAV load material on 0.1 ml CIMac-QA column. FIGS. 5A and 5C show chromatograms depicting the gradient elution profile AEX-WPC-MCC run on 0.1 mL CIMac-QA column of low-end loading (FIG. 5A) and high-end loading (FIG. 5C) respectively, using 11% full rAAV load material. The loading phase UV280 (mAU) traces of four AEX runs within one MCC loop are shown by a thin line (Run1), thick line (Run2), dashed line (Run3), and dot-dash line (Run4), respectively. FIGS. 5B and 5D show chromatograms depicting gradient elution profile of low-end loading AEX-WPC-MCC run on 0.1 mL CIMac-QA column of low-end loading (FIG. 5B) and high-end loading (FIG. 5D) respectively, using 11% full rAAV load material. The conductivity (mS/cm) trace is shown by the long-dash line. With conductivity increase, empty rAAV peak (denoted as “E”), full rAAV peak (denoted as “F”), and third peak (denoted as “T”) were sequentially resolved in salt linear gradient. The gradient elution phase UV280 (mAU) traces of four AEX runs within one MCC loop are shown in brown (Run1), blue (Run2), red (Run3), and purple (Run4), respectively. FIG. 5E is a comparison of the gradient elution profiles between low-end loading and high-end loading AEX-WPC-MCC runs using 11% Full rAAV Load Material on 0.1 m>CIMac-QA column. The UV280 (mAU) and UV254 (mAU) traces for high-end loading AEX-WPC-MCC run are shown by a thick solid line and a thin solid line, respectively. The UV280 (mAU) and UV254 (mAU) traces for low-end loading AEX-WPC-MCC run are shown by a dashed line and a dot-dash line, respectively. The conductivity (mS/cm) trace is shown by the long-dash line. Empty rAAV peak (denoted as “E”), full rAAV peak (denoted as “F”), and third peak (denoted as “T”) are indicated by the black arrows. FIG. 5F is a graph depicting the relationship between viral genome recovery and pool full rAAV % of AEX runs using 11% full rAAV load material on 0.1 mL CIMac-QA column. The normal batch loading run, low-end loading AEX-WPC-MCC run, and high-end loading AEX-WPC-MCC runs are shown in a tightly dotted circle, a loosely dotted circle, and an open circle, respectively.

FIG. 6A and FIG. 6B are bar graphs comparing the full viral capsid yield and separation of empty and full AAV capsid particles from the capsid preparation/loading material, using normal AEX or AEX-WPC-MCC runs. FIG. 6A shows graphs comparing AEX step yield of full rAAV from two separate rAAV capsid preparations, AAV prep 1 (upper panel) and AAV prep 2 (lower panel) based on viral genome recovery (y-axis) between normal AEX and AEX-WPC-MCC runs (x-axis). FIG. 6B shows graphs comparing AEX pool % recovery of empty (E), intermediate (I) and full (F) rAAV (Y-axis) capsids from two separate rAAV capsid preparations, rAAV prep 1 (upper panel) and rAAV prep 2 (lower panel) between normal AEX and AEX-WPC-MCC runs (x-axis).

FIG. 7 is a chart comparing the projected processing characteristics and yield at scale for both AEX-WPC-MCC run and AEX normal run based on their small-scale performance. The various processing characteristics (total processing time, column volume, total buffer usage, cycle, and volumetric loading) and yield (viral genome recovery, pool fill rAAV and productivity) of both AEX-WPC-MCC run and AEX normal run are as indicated. Increase or decrease in values of processing characteristics and yield between AEX-WPC-MCC run and AEX normal run are indicated by up and down arrows, respectively.

FIG. 8. is a graph depicting the distribution of host cell protein (HCP) in the AEX salt gradient elution. The resolved and fractionated UV peaks (based on the ratio of UV254/UV280) obtained from separation of empty and full AAV capsid particles from the capsid preparation/loading material using a combination of AEX-WPC-MCC run and gradient salt elution, are identified as empty peak (denoted as E), full peak (denoted as F), and third peak (denoted as T). Each fraction's HCP concentration is denoted by open circles, with values that were below the limit of detection labeled by a “<” sign.

### DETAILED DESCRIPTION OF CERTAIN EMBODIMENTS

### Definitions

Where the use of the term “about” is before a quantitative value, the disclosure encompasses the specific quantitative value itself, unless specifically stated otherwise. As used herein, the term “about” refers to a ±10% variation from the nominal value unless otherwise indicated or inferred.

As used herein, the term “adeno-associated virus” refers to a small, replication-defective, non-enveloped virus that infects humans and some other primate species. AAV is not known to cause disease and elicits a very mild immune response. Gene therapy vectors that utilize AAV can infect both dividing and quiescent cells and can persist in an extrachromosomal state without integrating into the genome of the host cell. These features make AAV an attractive viral vector for gene therapy. There are currently 13 recognized serotypes of AAV (AAV1-13).

Unless otherwise noted, where the term “between” is used to refer to a numerical range, the range includes the specified endpoints. For example, the range “between 1 mM and 10 mM” includes 1 mM, 10 mM, and values greater than 1 mM but less than 10 mM.

As used herein, the term “capsid particle” refers to a particle that comprises at least one viral capsid protein which (i) encapsidates a nucleic acid, e.g., a vector genome or a portion thereof, and/or (ii) forms a structure surrounding a core. In the case of empty capsid particles, as described herein, the core may be empty or collapsed, or may contain only a portion of a vector genome or may contain a portion of the host cell DNA. In some embodiments, a capsid particle encapsidates a nucleic acid that is a vector genome and/or gene of interest. In some embodiments, a capsid particle encapsidates a nucleic acid species that is not a vector genome or gene of interest, e.g., plasmid or host cell DNA, or a portion thereof.

As used herein, the term “full capsid particle” refers to a capsid particle that comprises a complete vector genome, that is, a vector genome that comprises a heterologous nucleic acid of interest flanked on both sides by AAV ITRs.

As used herein, the term “empty capsid particle,” refers to a capsid particle that includes at least one capsid protein and lacks a complete vector genome, e.g., the lacks in whole or in part, a heterologous nucleic acid of interest flanked on either side by AAV ITRs, or lacks in whole or in part, another part of the vector genome.

As used herein, the terms “gradient elution” or “gradient separation” refer to a mode of chromatographic separation wherein the concentration of one or more salts in the elution solution that is applied to the separation medium is gradually changed during the separation.

As used herein, the term “inverted terminal repeat” (abbreviated “ITR”) refers to a symmetrical nucleic acid sequences in the genome of adeno-associated viruses required for efficient replication. ITR sequences are located at each end of the AAV DNA genome. The ITRs serve as the origins of replication for viral DNA synthesis and are essential cis components for generating AAV integrating vectors.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

As used herein, the terms “isocratic elution” or “isocratic separation” refer to a mode of chromatographic separation where the concentration of all salts in the solution is kept constant during a defined period of the separation (e.g., the “wash” solution during a “wash” step and the “elution” solution during an “elution” step). In some embodiments, an isocratic elution uses a series of two or more separate solutions during the separation, each of which may have different fixed concentrations of one or more salts relative to another solution in the series.

As used herein, the phrase “isocratic elution gradient” refers to a gradient wherein the composition of the mobile phase is changed in steps during a single chromatographic run. In each step of an isocratic elution gradient, the mobile phase is kept at the same composition (e.g., constant concentration) until a subsequent step in the chromatographic run, at which time the composition of the mobile phase is changed such that the concentration of a component of the mobile phase is increased relative to the concentration of the component in a previous step. Thus, for example, an isocratic elution gradient of MgCl₂ involves using various steps, with a constant MgCl₂ concentration at each individual step, but with increasing MgCl₂ concentrations from one step to a subsequent step.

As used herein, the terms “normal batch loading,” “normal batch run,” “normal batch loading run,” and the like, refer to an AEX process for separating empty and full viral capsid particles using a rAAV preparation (loading material) comprising less than about 1×10¹⁴ vp/ml-column As used herein, the terms “batch loading” or “batch run” refer to an AEX process for separating empty and full viral capsid particles using a rAAV preparation (loading material) comprising passing the rAAV preparation (loading material) through a single AEX chromatography medium/column. Alternately, the terms “normal batch loading,” “normal batch run,” “normal batch loading run,” and the like, refer to an AEX process for separating empty and full viral capsid particles using a rAAV preparation (loading material) comprising less than about 1×10¹⁴ vp/ml-column through a single AEX chromatography column, and wherein the process comprises passing the rAAV preparation (loading material) through a single AEX chromatography medium/column. As used herein, the terms “normal batch loading,” “normal batch run,” “normal batch loading run,” and the like, refer to an AEX process for separating empty and full viral capsid particles using a rAAV preparation (loading material) without using a multi-column chromatography method.

As used herein, by “obtaining” or “to obtain” with respect to a “fraction”, “eluent”, “flowthrough”, or “wash fraction”—e.g., an eluent after a step of passing through or applying an elution solution to an anion exchange medium—it is meant that the fraction or eluent is generated during and until the end of that step. A fraction that is “obtained” or “generated” may or may not be collected.

As used herein, the term “quaternary ammonium salt” refers to an ionic compound having a quaternary ammonium nitrogen, four groups (e.g., alkyl or aryl groups) connected to the ammonium nitrogen, and an anionic ion (e.g., acetate, bromide, or chloride). The quaternary ammonium salt can be a tetraalkylammonium salt, e.g., a tetraalkylammonium chloride or a tetraalkylammonium acetate. In some embodiments, the quaternary ammonium salt is a tetraalkylammonium chloride selected from the group consisting of tetramethylammonium chloride (TMAC), tetraethylammonium chloride (TEAC), tetrapropylammonium chloride (TPAC), tetrabutylammonium chloride (TBAC), benzyltributylammonium chloride (BTBAC), or any combination(s) thereof. The quaternary ammonium salt can be a tetraalkylammonium acetate selected from the group consisting of tetramethylammonium acetate, tetraethylammonium acetate (TEA-Ac), tetrapropylammonium acetate, tetrabutylammonium acetate, and any combination(s) thereof.

As used herein, the term “recombinant,” may be used to describe, e.g., a nucleic acid molecule that has a sequence that is not naturally occurring or has a sequence that is made by an artificial combination of two otherwise separated segments of sequence. This artificial combination can be accomplished by chemical synthesis or by the artificial manipulation of isolated segments of nucleic acid molecules, such as by genetic engineering techniques.

A “recombinant adeno-associated virus preparation” or “rAAV preparation,” refers to a product that results from a method of manufacturing recombinant AAV in a host cell (e.g., in a mammalian cell or an insect cell). In some embodiments, a recombinant AAV preparation includes a mixture of full rAAV particles and empty rAAV particles. In some embodiments, a recombinant AAV preparation has been subjected to one or more downstream operations after initial upstream operations, e.g., nuclease treatment, filtration to remove host-cell impurities, and/or affinity purification using ligands that bind AAV capsids, as well known to those of skill in the art.

As used herein, the term “salt composition,” when used in reference to a solution, refers to the identities and amounts of all salts in that solution. Thus, if a solution's “salt composition” is said to remain constant throughout a particular duration, it is meant that the identities and amounts of all salts in that solution remain constant throughout the particular duration.

As used herein, the term “separation chemistry” refers to the active ligand, such as quaternary amine or mixed and supporting matrix of the separation medium.

As used herein, the terms “separation medium,” “medium,” “chromatography medium,” and the like, refer to a physical structure, such as column packed with resins or a monolith or a membrane, to which a rAAV preparation is applied in order to achieve separation of certain fractions of the preparation. For example, a rAAV preparation may be applied to a column, which column is then washed with one or more solutions to separate (and collect separated fractions) empty and full-AAV particles from one another. In some embodiments, a separation medium is an anion-exchange medium. In some embodiments, a separation medium is a mixed-modal medium that can serve as an anion-exchange medium. In some embodiments, a separation medium is a column (e.g., a monolithic column or particles in a packed column). In some embodiments, a separation medium is a membrane.

As used herein, the term “vector” is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An expression vector is a vector that contains the necessary regulatory sequences to allow transcription and translation of inserted gene or genes. In some embodiments herein, the vector is an AAV vector.

### Separation Methods

The present disclosure provides improved methods for separating full viral capsid particles from empty capsid particles in a viral capsid preparation or viral capsid populations comprising both empty and full capsid particles.

In some embodiments, the present disclosure provides a method of separating empty capsid particles from full capsid particles in a viral capsid preparation comprising empty and full capsid particles, the method comprising the steps of: a) applying an equilibrating solution to at least one anion exchange (AEX) medium; and b) applying a volume of the viral capsid preparation to the at least one AEX medium, and passing the volume of the viral capsid preparation through the at least one AEX medium to generate a flowthrough comprising empty capsid particles. In some embodiments, the present disclosure provides a method of separating empty capsid particles from full capsid particles in a viral capsid preparation comprising empty and full capsid particles, the method comprising the steps of: a) applying an equilibrating solution to at least one anion exchange (AEX) medium; and b) passing a volume of the viral capsid preparation through the at least one AEX medium to generate a flowthrough comprising empty capsid particles. In some embodiments, the volume of the viral capsid preparation comprises between 1×10¹⁴ to 5.5×10¹⁵ (e.g., 1×10¹⁴ to 1.5×10¹⁴, 1.5×10¹⁴ to 2×10¹⁴, 2×10¹⁴ to 2.5×10¹⁴, 2.5×10¹⁴ to 3×10¹⁴, 3×10¹⁴ to 3.5×10¹⁴, 3.5×10¹⁴ to 4×10¹⁴, 4×10¹⁴ to 4.5×10¹⁴, 4.5×10¹⁴ to 5×10¹⁴, 5×10¹⁴ to 5.5×10¹⁴, 5.5×10¹⁴ to 6.5×10¹⁴, 6.5×10¹⁴ to 7.5×10¹⁴, 7.5×10¹⁴ to 8.5×10¹⁴, 8.5×10¹⁴ to 9.5×10¹⁴, 9.5×10¹⁴ to 1×10¹⁵, 1×10¹⁵ to 1.5×10¹⁵, 1.5×10¹⁵ to 2×10¹⁵, 2×10¹⁵ to 2.5×10¹⁵, 2.5×10¹⁵ to 3×10¹⁵, 3×10¹⁵ to 3.5×10¹⁵, 3.5×10¹⁵ to 4×10¹⁵, 4×10¹⁵ to 4.5×10¹⁵, 5×10¹⁵ to 5.5×10¹⁵, 1.5×10¹⁴×5×10¹⁵, 2×10¹⁴ to 4.5×10¹⁵, 2.5×10¹⁴ to 4×10¹⁵, 3×10¹⁴ to 3.5×10¹⁵, 3.5×10¹⁴ to 2.5×10¹⁵, 4×10¹⁴ to 2×10¹⁵, 4.5×10¹⁴ to 1.5×10¹⁵, 5×10¹⁴ to 1×10¹⁵, 5.5×10¹⁴ to 9.5×10¹⁴, 6.5×10¹⁴ to 8.5×10¹⁴, 6.5×10¹⁴ to 5×10¹⁵, 7.5×10¹⁴ to 4.5×10¹⁵, 5.5×10¹⁴ to 5.5×10¹⁵, 6.5×10¹⁴ to 3.5×10¹⁵, 8.5×10¹⁴ to 4×10¹⁵, 9.5×10¹⁴ to 3.5×10¹⁵, 1×10¹⁵ to 3×10¹⁵, or 1×10¹⁵ to 2×10¹⁵ and all integers including and in between 1×10¹⁴ to 5.5×10¹⁵) virus particles per milliliter of the AEX medium (expressed as “vp/mL” or “vp/ml-column” herein).

In some embodiments, the present disclosure also provides a method of separating full capsid particles and empty capsid particles in a viral capsid preparation comprising empty and full capsid particles, the method comprising the steps of: a) applying an equilibrating solution to at least one anion exchange (AEX) medium; b) applying a volume of the viral capsid preparation to the at least one AEX medium and passing the volume of the viral capsid preparation through the at least one AEX medium to generate a flowthrough comprising empty capsid particles; and c) applying an elution solution comprising a salt to the at least one AEX medium and passing the elution solution through the at least one AEX medium to generate an eluent comprising full capsid particles. In some embodiments, the present disclosure provides a method of separating full capsid particles and empty capsid particles in a viral capsid preparation comprising empty and full capsid particles, the method comprising the steps of a) applying an equilibrating solution to at least one anion exchange (AEX) medium; b) passing a volume of the viral capsid preparation through the at least one AEX medium to generate a flowthrough comprising empty capsid particles; and c) passing an elution solution through the at least one AEX medium to generate an eluent comprising full capsid particles. In some embodiments, the volume of the viral capsid preparation comprises between 1×10¹⁴ to 5.5×10¹⁵ (e.g., 1×10¹⁴ to 1.5×10¹⁴, 1.5×10¹⁴ to 2×10¹⁴, 2×10¹⁴ to 2.5×10¹⁴, 2.5×10¹⁴ to 3×10¹⁴, 3×10¹⁴ to 3.5×10¹⁴, 3.5×10¹⁴ to 4×10¹⁴, 4×10¹⁴ to 4.5×10¹⁴, 4.5×10¹⁴ to 5×10¹⁴, 5×10¹⁴ to 5.5×10¹⁴, 5.5×10¹⁴ to 6.5×10¹⁴, 6.5×10¹⁴ to 7.5×10¹⁴, 7.5×10¹⁴ to 8.5×10¹⁴, 8.5×10¹⁴ to 9.5×10¹⁴, 9.5×10¹⁴ to 1×10¹⁵, 1×10¹⁵ to 1.5×10¹⁵, 1.5×10¹⁵ to 2×10¹⁵, 2×10¹⁵ to 2.5×10¹⁵, 2.5×10¹⁵ to 3×10¹⁵, 3×10¹⁵ to 3.5×10¹⁵, 3.5×10¹⁵ to 4×10¹⁵, 4×10¹⁵ to 4.5×10¹⁵, 5×10¹⁵ to 5.5×10¹⁵, 1.5×10¹⁴×5×10¹⁵, 2×10¹⁴ to 4.5×10¹⁵, 2.5×10¹⁴ to 4×10¹⁵, 3×10¹⁴ to 3.5×10¹⁵, 3.5×10¹⁴ to 2.5×10¹⁵, 4×10¹⁴ to 2×10¹⁵, 4.5×10¹⁴ to 1.5×10¹⁵, 5×10¹⁴ to 1×10¹⁵, 5.5×10¹⁴ to 9.5×10¹⁴, 6.5×10¹⁴ to 8.5×10¹⁴, 6.5×10¹⁴ to 5×10¹⁵, 7.5×10¹⁴ to 4.5×10¹⁵, 5.5×10¹⁴ to 5.5×10¹⁵, 6.5×10¹⁴ to 3.5×10¹⁵, 8.5×10¹⁴ to 4×10¹⁵, 9.5×10¹⁴ to 3.5×10¹⁵, 1×10¹⁵ to 3×10¹⁵, or 1×10¹⁵ to 2×10¹⁵ and all integers including and in between 1×10¹⁴ to 5.5×10¹⁵) virus particles per milliliter of the AEX medium.

In some embodiments, the present disclosure also provides a method of increasing the percentage of full capsid particles in a viral capsid preparation comprising empty and full capsid particles, the method comprising the steps of: a) applying an equilibrating solution to at least one anion exchange (AEX) medium; b) applying a volume of the viral capsid preparation to the at least one AEX medium, and passing the volume of the viral capsid preparation through the at least one AEX medium to generate a flowthrough; c) applying an elution solution comprising a salt to the at least one AEX medium and passing the elution solution through the at least one AEX medium to generate an eluent; and d) collecting the eluent from step c), wherein a volume of the eluent of step d) comprises full capsid particles at a percentage that is higher than the percentage of full capsid particles in an equivalent volume of the viral capsid preparation. In some embodiments, the present disclosure provides a method of increasing the percentage of full capsid particles in a viral capsid preparation comprising empty and full capsid particles, the method comprising the steps of a) applying an equilibrating solution to at least one anion exchange (AEX) medium; b) passing a volume of the viral capsid preparation through the at least one AEX medium to generate a flowthrough; c) passing an elution solution comprising a salt through the at least one AEX medium to generate an eluent; and d) collecting the eluent from step c), wherein a volume of the eluent of step d) comprises full capsid particles at a percentage that is higher than the percentage of full capsid particles in an equivalent volume of the viral capsid preparation. In some embodiments, the volume of the viral capsid preparation comprises between 1×10¹⁴ to 5.5×10¹⁵ (e.g., 1×10¹⁴ to 1.5×10¹⁴, 1.5×10¹⁴ to 2×10¹⁴, 2×10¹⁴ to 2.5×10¹⁴, 2.5×10¹⁴ to 3×10¹⁴, 3×10¹⁴ to 3.5×10¹⁴, 3.5×10¹⁴ to 4×10¹⁴, 4×10¹⁴ to 4.5×10¹⁴, 4.5×10¹⁴ to 5×10¹⁴, 5×10¹⁴ to 5.5×10¹⁴, 5.5×10¹⁴ to 6.5×10¹⁴, 6.5×10¹⁴ to 7.5×10¹⁴, 7.5×10¹⁴ to 8.5×10¹⁴, 8.5×10¹⁴ to 9.5×10¹⁴, 9.5×10¹⁴ to 1×10¹⁵, 1×10¹⁵ to 1.5×10¹⁵, 1.5×10¹⁵ to 2×10¹⁵, 2×10¹⁵ to 2.5×10¹⁵, 2.5×10¹⁵ to 3×10¹⁵, 3×10¹⁵ to 3.5×10¹⁵, 3.5×10¹⁵ to 4×10¹⁵, 4×10¹⁵ to 4.5×10¹⁵, 1×10¹⁵ to 5.5×10¹⁵, 1.5×10¹⁴×5×10¹⁵, 2×10¹⁴ to 4.5×10¹⁵, 2.5×10¹⁴ to 4×10¹⁵, 3×10¹⁴ to 3.5×10¹⁵, 3.5×10¹⁴ to 2.5×10¹⁵, 4×10¹⁴ to 2×10¹⁵, 4.5×10¹⁴ to 1.5×10¹⁵, 5×10¹⁴ to 1×10¹⁵, 5.5×10¹⁴ to 9.5×10¹⁴, 6.5×10¹⁴ to 8.5×10¹⁴, 6.5×10¹⁴ to 5×10¹⁵, 7.5×10¹⁴ to 4.5×10¹⁵, 5.5×10¹⁴ to 5.5×10¹⁵, 6.5×10¹⁴ to 3.5×10¹⁵, 8.5×10¹⁴ to 4×10¹⁵, 9.5×10¹⁴ to 3.5×10¹⁵, 1×10¹⁵ to 3×10¹⁵, or 1×10¹⁵ to 2×10¹⁵ and all integers including and in between 1×10¹⁴ to 5.5×10¹⁵) vp/ml of the AEX medium.

In some embodiments, step b) further comprises collecting at least a portion of the flowthrough and analyzing the empty and full capsid content of the collected portion of the flowthrough, wherein the applying or passing of the volume of the viral capsid preparation to or through the at least one AEX-medium in step b) is stopped if full capsid particle is detected in the collected portion of the flowthrough.

In some embodiments of the methods of the disclosure, the applying or passing of the volume of the viral capsid preparation to or through the at least one AEX-medium in step b) is stopped if ≤5% (e.g., 5%, 4%, 3%, 2%, 1%, 0.5%, 0.25% or 0%, and all % amounts including and in between 0% to 5%) full capsid particle is detected in the collected portion of the flowthrough. In some embodiments of the method of the disclosure, the applying or passing of the volume of the viral capsid preparation to or through the at least one AEX-medium in step b) is stopped if 5% full capsid particle is detected in the collected portion of the flowthrough.

In some embodiments of the methods of the disclosure, a UV spectrophotometry method is used to assess a viral capsid preparation and/or a fraction (e.g., an elution fraction). For example, the amount of light at wavelengths at or around 254 nm and/or 260 nm that is absorbed by sample is generally proportional to the concentration of nucleic acids in the sample. Additionally, proteins have a greater absorbance at 280 nm (A280) than they do at 254 nm (A254) or 260 nm (A260); the inverse is true for nucleic acids, which have a greater A254 or A260 than A280. Thus, full capsid particles, which have a greater amount of DNA than do empty capsid particles, will have a greater A254/A280 or A260/A280 ratio than would empty capsid particles. This difference can be exploited to assess the relative amounts of full and empty particles in a sample. In some embodiments, one or more of A254, A260, A280, A254/A280 ratio, or A260/A280 ratio, is assessed.

In some embodiments of the methods of the disclosure, the analyzing the empty and full capsid content comprises determining the A254/A280 ratio of the collected portion of the flowthrough using UV spectrophotometry. In some embodiments, the A254/A280 ratio is directly proportional to the presence of full capsid particles in a sample, e.g., the flowthrough.

In some embodiments, the methods of the disclosure comprise using at least two, at least three or at least four AEX media. In some embodiments, the methods of the disclosure comprise using two AEX media. In some embodiments, the methods of the disclosure comprise using three AEX media. In some embodiments, the methods of the disclosure comprise using four AEX media.

In some embodiments of the methods of the disclosure, the volume of the viral capsid preparation applied in step b) is constant between each of the at least two, at least three, or at least four AEX media.

In some embodiments, the methods of the disclosure comprise sequential or simultaneous use of the at least two, at least three or at least four AEX media simultaneously or sequentially. In some embodiments, the methods of the disclosure comprise sequential use of the at least two, at least three or at least four AEX media.

In some embodiments, the sequential use comprises completing each of the steps using one AEX medium, then repeating each of the steps starting with step a) using a subsequent AEX medium.

In some embodiments, the sequential use comprises completing at least one of the steps with one AEX medium, then performing each of the steps starting with step a) using a subsequent AEX medium. In some embodiments, the sequential use comprises completing applying a volume of the capsid preparation to the at least one AEX medium, then conducting step a) using a subsequent AEX medium. In some embodiments, the sequential use comprises completing generating a flowthrough from the at least one AEX medium, then conducting step a) using a subsequent AEX medium.

In some embodiments, the sequential use comprises completing applying an elution solution to the at least one AEX medium, then conducting step a) using a subsequent AEX media. In some embodiments, the sequential use comprises completing generating an eluent from the at least one AEX medium, then conducting step a) using a subsequent AEX medium.

In some embodiments, the methods of the present disclosure comprise repeating each of the steps at least one time (e.g., 1 time, 2 times, 3 times, 4 times, 5 times, 6 times, 7 times, 8 times, 9 times, 10 times or more), each time with a different AEX medium. In some embodiments, the methods of the present disclosure comprise repeating each of the steps two times, each time with a different AEX medium. In some embodiments, the methods of the present disclosure comprise repeating each of the steps three times, each time with a different AEX medium. In some embodiments, the methods of the present disclosure comprise repeating each of the steps four times, each time with a different AEX medium.

In some embodiments, the volume of the viral capsid preparation applied to the at least one AEX medium remains constant between each repetition (e.g., one, two, three, four, five, six, seven, eight, nine or ten repetition(s)) of the method. In some embodiments, the volume of the viral capsid preparation applied to the at least one AEX medium does not remain constant between each repetition. In some embodiments, the volume of the viral capsid preparation applied to the at least one AEX medium in each repeat is higher than the volume of the viral capsid preparation applied to the at least one AEX medium the first time or in the previous repeat.

In some embodiments, the volume of the viral capsid preparation applied to the at least one AEX medium in each repetition is lower than the volume of the viral capsid preparation applied to the at least one AEX medium the first time or in the previous repetition. In some embodiments, the volume of the viral capsid preparation applied to the at least one AEX medium increases linearly with each repetition. In some embodiments, the volume of the viral capsid preparation applied to the at least one AEX medium increases stepwise with each repetition. In some embodiments, the volume of the viral capsid preparation applied to the at least one AEX medium decreases linearly with each repetition. In some embodiments, the volume of the viral capsid preparation applied to the at least one AEX medium decreases stepwise with each repetition.

In some embodiments in which steps are repeated, the methods comprise passing at least a first wash solution (e.g., at least one wash solution, a least two wash solutions, at least three wash solutions, at least four wash solutions, at least five wash solutions or more) through the at least one AEX medium between each repetition.

In some embodiments, the present disclosure also provides an elution method comprising: a) contacting at least one anion exchange (AEX) medium with a volume of a viral capsid preparation comprising full capsid particles and empty capsid particles and passing the volume of viral capsid preparation through the AEX medium to generate a flowthrough comprising empty capsid particles; and b) contacting the at least one AEX medium an elution solution comprising a salt and passing the elution solution through the at least one AEX medium to generate an eluent comprising full capsid particles. In some embodiments, the present disclosure also provides an elution method comprising: a) passing a volume of viral capsid preparation comprising full capsid particles and empty capsid particles through an AEX medium to generate a flowthrough comprising empty capsid particles; and b) passing an elution solution comprising a salt through the at least one AEX medium to generate an eluent comprising full capsid particles. In some embodiments, the volume of the viral capsid preparation comprises between 1×10¹⁴ to 5.5×10¹⁵ (e.g., 1×10¹⁴ to 1.5×10¹⁴, 1.5×10¹⁴ to 2×10¹⁴, 2×10¹⁴ to 2.5×10¹⁴, 2.5×10¹⁴ to 3×10¹⁴, 3×10¹⁴ to 3.5×10¹⁴, 3.5×10¹⁴ to 4×10¹⁴, 4×10¹⁴ to 4.5×10¹⁴, 4.5×10¹⁴ to 5×10¹⁴, 5×10¹⁴ to 5.5×10¹⁴, 5.5×10¹⁴ to 6.5×10¹⁴, 6.5×10¹⁴ to 7.5×10¹⁴, 7.5×10¹⁴ to 8.5×10¹⁴, 8.5×10¹⁴ to 9.5×10¹⁴, 9.5×10¹⁴ to 1×10¹⁵, 1×10¹⁵ to 1.5×10¹⁵, 1.5×10¹⁵ to 2×10¹⁵, 2×10¹⁵ to 2.5×10¹⁵, 2.5×10¹⁵ to 3×10¹⁵, 3×10¹⁵ to 3.5×10¹⁵, 3.5×10¹⁵ to 4×10¹⁵, 4×10¹⁵ to 4.5×10¹⁵, 5×10¹⁵ to 5.5×10¹⁵, 1.5×10¹⁴×5×10¹⁵, 2×10¹⁴ to 4.5×10¹⁵, 2.5×10¹⁴ to 4×10¹⁵, 3×10¹⁴ to 3.5×10¹⁵, 3.5×10¹⁴ to 2.5×10¹⁵, 4×10¹⁴ to 2×10¹⁵, 4.5×10¹⁴ to 1.5×10¹⁵, 5×10¹⁴ to 1×10¹⁵, 5.5×10¹⁴ to 9.5×10¹⁴, 6.5×10¹⁴ to 8.5×10¹⁴, 6.5×10¹⁴ to 5×10¹⁵, 7.5×10¹⁴ to 4.5×10¹⁵, 5.5×10¹⁴ to 5.5×10¹⁵, 6.5×10¹⁴ to 3.5×10¹⁵, 8.5×10¹⁴ to 4×10¹⁵, 9.5×10⁴ to 3.5×10¹⁵, 1×10¹⁵ to 3×10¹⁵, or 1×10¹⁵ to 2×10¹⁵ and all integers including and in between 1×10¹⁴ to 5.5×10¹⁵) vp/ml of the AEX medium.

In some embodiments of the elution methods of the present disclosure, step a) further comprises collecting at least a portion of the flowthrough and analyzing the empty and full capsid content of the collected portion of the flowthrough, wherein the contacting or passing of the volume of the viral capsid preparation to the at least one AEX medium in step a) is stopped if full capsid particles are detected in the collected portion of the flowthrough.

In some embodiments of the elution methods of the disclosure, the passing of the volume of the viral capsid preparation to the at least one AEX-medium in step a) is stopped if ≤5% (e.g., 5%, 4%, 3%, 2%, 1%, 0.5%, 0.25% or 0%, and all % amounts between and including 05 to 5%) full capsid particles are detected in the collected portion of the flowthrough. In some embodiments, the passing of the volume of the viral capsid preparation to the at least one AEX-medium in step a) is stopped if 5% full capsid particles are detected in the flowthrough.

In some embodiments of the elution methods of the disclosure, a UV spectrophotometry method is used to assess a viral capsid preparation and/or a fraction (e.g., an elution fraction). For example, the amount of light at wavelengths at or around 254 nm and/or 260 nm that is absorbed by sample is generally proportional to the concentration of nucleic acids in the sample. Additionally, proteins have a greater absorbance at 280 nm (A280) than they do at 254 nm (A254) or 260 nm (A260); the inverse is true for nucleic acids, which have a greater A254 or A260 than A280. Thus, full capsid particles, which have a greater amount of DNA than do empty capsid particles, will have a greater A254/A280 or A260/A280 ratio than would empty capsid particles. This difference can be exploited to assess the relative amounts of full and empty particles in a sample. In some embodiments, one or more of A254, A260, A280, A254/A280 ratio, or A260/A280 ratio, is assessed.

In some embodiments of the elution methods of the disclosure, analyzing the empty and full capsid content comprises determining the A254/A280 ratio using UV spectrophotometry, wherein the ratio of O.D. at 254 nm/280 nm is directly proportional to the presence of full capsid particles in the flowthrough.

In some embodiments, the elution methods of the disclosure comprise using at least two, at least three, or at least four AEX media. In some embodiments, the elution methods of the disclosure, comprise using two AEX media. In some embodiments, the elution methods of the disclosure comprise using three AEX media. In some embodiments, the elution methods of the disclosure comprise using four AEX media.

In some embodiments of the elution methods of the disclosure, the volume of the viral capsid preparation contacted with the at least one AEX medium in step a) is constant between each of the at least two, at least three or at least four AEX media.

In some embodiments, the elution methods of the disclosure comprise simultaneous or sequential use of the at least two, at least three or at least four AEX media. In some embodiments, the elution methods of the disclosure comprise sequential use of the at least two, at least three or at least four AEX media.

In some embodiments, the sequential use comprises completing each of the steps using one AEX medium, then repeating each of the steps starting with step a) using a subsequent AEX medium.

In some embodiments, the sequential use comprises completing at least one of the steps, then performing each of the steps starting with step a) using a subsequent AEX medium. In some embodiments, the sequential use comprises completing applying a volume of the capsid preparation to the at least one AEX medium, then conducting step a) using a subsequent AEX medium. In some embodiments, the sequential use comprises completing generating a flowthrough from the at least one AEX medium, then conducting of step a) using a subsequent AEX medium.

In some embodiments, the sequential use comprises completing applying an elution solution to the at least one AEX medium, then conducting step a) using a subsequent AEX medium. In some embodiments, the sequential use comprises completing generating an eluent from the at least one AEX medium, then the conducting of the step a) using a subsequent AEX medium.

In some embodiments, the elution methods of the present disclosure comprise repeating each of the steps at least one time (e.g., 1 time, 2 times, 3 times, 4 times, 5 times, 6 times, 7 times, 8 times, 9 times, 10 times or more) each time with a different AEX medium. In some embodiments, the elution methods of the present disclosure comprise repeating each of the steps, two times, each time with a different AEX medium. In some embodiments, the elution methods of the present disclosure comprise repeating each of steps three times, each time with a different AEX medium. In some embodiments, the elution methods of the present disclosure comprise repeating each of steps four times, each time using a different AEX medium.

In some embodiments of the elution methods of the present disclosure, the volume of the viral capsid preparation applied to the at least one AEX medium remains constant between each repetition (e.g., one, two, three, four, five, six, seven, eight, nine or ten repeat(s)). In some embodiments of the elution methods of the present disclosure, the volume of the viral capsid preparation applied to the at least one AEX medium does not remain constant between each repetition. In some embodiments of the elution methods of the present disclosure, the volume of the viral capsid preparation applied to the at least one AEX medium in each repeat is higher than the volume of the viral capsid preparation applied to the at least one AEX medium the first time or in the previous repetition.

In some embodiments of the elution methods of the present disclosure, the volume of the viral capsid preparation applied to the at least one AEX medium in each repetition is lower than the volume of the viral capsid preparation applied to the at least one AEX medium the first time or in the previous repetition. In some embodiments of the elution methods of the present disclosure, the volume of the viral capsid preparation applied to the at least one AEX medium increases linearly with each repetition. In some embodiments of the elution methods of the present disclosure, the volume of the viral capsid preparation applied to the at least one AEX medium increases stepwise with each repetition. In some embodiments of the elution methods of the present disclosure, the volume of the viral capsid preparation applied to the at least one AEX medium decreases linearly with each repetition. In some embodiments, the volume of the viral capsid preparation applied to the at least one AEX medium decreases stepwise with each repetition.

In some embodiments, the elution methods of the present disclosure comprising repeating each of steps of the disclosed method, comprise passing at least a first wash solution (e.g., at least one wash solution, at least two wash solutions, at least three wash solutions, at least four wash solutions, at least five wash solutions or more) through the at least one AEX medium between each repetition.

### Viral Capsid Preparation and Flowthrough

Methods of the present disclosure generally comprise a step of applying to or contacting with at least one anion exchange medium a volume of a viral capsid preparation comprising full capsid particles and empty capsid particles and passing the viral capsid preparation through the anion exchange medium to generate a flowthrough.

In some embodiments of the methods of the present disclosure, the volume of the viral capsid preparation applied to the AEX medium comprises between 5.5×10¹⁴ to 5.5×10¹⁵ (e.g., 5.5×10¹⁴ to 6.5×10¹⁴, 6.5×10¹⁴ to 7.5×10¹⁴, 7.5×10¹⁴ to 8.5×10¹⁴, 8.5×10¹⁴ to 9.5×10¹⁴, 9.5×10¹⁴ to 1×10¹⁵, 1×10¹⁵ to 1.5×10¹⁵, 15×10¹⁵ to 2×10¹⁵, 2×10¹⁵ to 3×10¹⁵, 3×10¹⁵ to 3.5×10¹⁵, 3.5×10¹⁵ to 4×10¹⁵, 4×10¹⁵ to 4.5×10¹⁵, 5×10¹⁵ to 5.5×10¹⁵, 6.5×10¹⁴ to 5×10¹⁵, 7.5×10¹⁴ to 4.5×10¹⁵, 8.5×10¹⁴ to 4×10¹⁵, 9.5×10¹⁴ to 3.5×10¹⁵, 1×10¹⁵ to 3×10¹⁵, or 1×10¹⁵ to 2×10¹⁵, and all integers including and in between 5.5×10¹⁴ to 5.5×10¹⁵) vp/ml of the AEX medium.

In some embodiments of the methods of the present disclosure, the volume of the viral capsid preparation applied to the AEX medium comprises between 6.7×10¹⁴ to 3.1×10¹⁵ (e.g., 6.7×10¹⁴ to 7.4×10¹⁴, 7.4×10¹⁴ to 8.1×10¹⁴, 8.1×10¹⁴ to 8.5×10¹⁴, 8.5×10¹⁴ to 9.2×10¹⁴, 9.2×10¹⁴ to 9.7×10¹⁴, 9.7×10¹⁴ to 1×10¹⁵, 1×10¹⁵ to 1.7×10¹⁵, 1.7×10¹⁵ to 2.4×10¹⁵ or 2.4×10¹⁵ to 3.1×10¹⁵) vp/ml of the AEX media.

In some embodiments of the methods of the present disclosure, the volume of the viral capsid preparation applied to the AEX medium comprises between 6.5×10¹⁴ to 5.5×10¹⁵ (6.5×10¹⁴ to 7×10¹⁴, 7×10¹⁴ to 7.5×10¹⁴, 7.5×10¹⁴ to 8×10¹⁴, 8×10¹⁴ to 8.5×10¹⁴, 8.5×10¹⁴ to 9×10¹⁴, 9×10¹⁴ to 9.5×10¹⁴, 9.5×10¹⁴ to 1×10¹⁵, 1×10¹⁵ to 1.5×10¹⁵, 1.5×10¹⁵ to 2×10¹⁵, 2×10¹⁵ to 2.5×10¹⁵, 2.5×10¹⁵ to 3.0×10¹⁵, 2.5×10¹⁵ to 3×10¹⁵, 3×10¹⁵ to 3.5×10¹⁵, 3.5×10¹⁵ to 4×10¹⁵, 4×10¹⁵ to 4.5×10¹⁵, 4.5×10¹⁵ to 5×10¹⁵, 5×10¹⁵ to 5.5×10¹⁵, 7×10¹⁴ to 5×10¹⁵, 7.5×10¹⁴ to 4.5×10¹⁵, 8×10¹⁴ to 4×10¹⁵, 8.5×10¹⁴ to 3.5×10¹⁵, 9×10¹⁴ to 2.5×10¹⁵, 9.5×10¹⁴ to 2×10¹⁵ or 2×10¹⁵ to 1.5×10¹⁵, and all integers including and in between 6.5×10¹⁴ to 5.5×10¹⁵) vp/ml of the AEX media.

In some embodiments of the methods of the present disclosure, the volume of the viral capsid preparation applied to the AEX medium comprises between 5.5×10¹⁴ to 3.5×10¹⁵ (e.g., 5.5×10¹⁴ to 6×10¹⁴, 6×10¹⁴ to 6.5×10¹⁴, 6.5×10¹⁴ to 7×10¹⁴, 7×10¹⁴ to 7.5×10¹⁴, 7.5×10¹⁴ to 8×10¹⁴, 8×10¹⁴ to 8.5×10¹⁴, 8.5×10¹⁴ to 9×10¹⁴, 9×10¹⁴ to 9.5×10¹⁴, 9.5×10¹⁴ to 1×10¹⁵, 1×10¹⁵ to 1.5×10¹⁵, 1.5×10¹⁵ to 2×10¹⁵, 2×10¹⁵ to 2.5×10¹⁵, 2.5×10¹⁵ to 3×10¹⁵, 3×10¹⁵ to 3.5×10¹⁵, 6×10¹⁴ to 3×10¹⁵, 6.5×10¹⁴ to 2.5×10¹⁵, 7×10¹⁴ to 2×10¹⁵, 7.5×10¹⁴ to 1.5×10¹⁵, 8×10¹⁴ to 1×10¹⁵, 8×10¹⁴ to 9×10¹⁴ or 8.5×10¹⁴ to 9.5×10¹⁴, and all integers including and in between 5.5×10¹⁴ to 3.5×10¹⁵) vp/ml of the AEX media. In some embodiments of the methods of the present disclosure, the volume of the viral capsid preparation applied to the AEX medium comprises between 7×10¹⁴ to 3×10¹⁵ (7×10¹⁴ to 7.5×10¹⁴, 7.5×10¹⁴ to 8×10¹⁴, 8×10¹⁴ to 8.5×10¹⁴, 8.5×10¹⁴ to 9×10¹⁴, 9×10¹⁴ to 9.5×10¹⁴, 9.5×10¹⁴ to 1×10¹⁵, 1×10¹⁵ to 1.5×10¹⁵, 1.5×10¹⁵ to 2×10¹⁵, 2×10¹⁵ to 2.5×10¹⁵, 2.5×10¹⁵ to 3×10¹⁵, 7.5×10¹⁴ to 2.5×10¹⁵, 8×10¹⁴ to 2×10¹⁵, 8.5×10¹⁴ to 1.5×10¹⁵, 9×10¹⁴ to 1×10¹⁵, 9.5×10¹⁴ to 1.5×10¹⁵, and all integers including and in between 7×10¹⁴ to 3×10¹⁵) vp/ml of the AEX media.

In some embodiments of the methods of the present disclosure, the viral capsid preparation comprises about 1% to about 60% (e.g., about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 5% to about 55%, about 10% to about 50%, about 15% to about 45%, about 20% to about 40%, about 25% to 35% and all % amounts including and in between about 1% to about 60%) full capsid particles. In some embodiments of the methods of the present disclosure, the viral capsid preparation comprises about 1% to about 40% (e.g., about 1% to about 5%, about 5% to about 10%, about 10% to about 15%, about 15% to about 20%, about 20% to about 25%, about 25% to about 30%, about 30% to about 35%, about 35% to about 40%, about 5% to about 35%, about 10% to about 30%, about 15% to about 25%, about 5% to about 15% and all % amounts including and in between about 1% to about 40%) full capsid particles. In some embodiments of the methods or elution method of the present disclosure, the viral capsid preparation comprises about 4% to about 11% (e.g., about 4% to about 5%, about 5% to about 6%, about 6% to about 7%, about 7% to about 8%, about 8% to about 9%, about 9% to about 10% or about 10% to about 110, about 5% to about 10%, about 6% to about 9%, about 7% to about 9%, about 5% to about 7%, about 4% to about 6%, about 6% to about 8%, about 8% to about 10% or about 95 to about 11%, and all % amounts including and in between about 4% to about 11%) full capsid particles. In some embodiments of the methods, the viral capsid preparation comprises about 4% full capsid particles. In some embodiments of the methods or elution method of the present disclosure, the viral capsid preparation comprises about 11% full capsid particles. In some embodiments, the flowthrough generated by passing a viral capsid preparation comprising through the at least one AEX medium can include both “empty capsid particles” and “intermediate capsid particles”. The term “intermediate capsid particle” as used herein refers to viral capsid particles comprising of fragments of host cell DNA (HCDNA), fragments or portions of the AAV genome or a combination thereof. Typically, the “empty capsid particles” and “intermediate capsid particles” can be released into the same flowthrough fractions. In some embodiments, the “intermediate capsid particles comprise between 0% to 40% of the flowthrough fractions comprising empty and intermediate capsid particles.

In some embodiments of the methods of the present disclosure, the viral capsid preparation comprises about 6.7×10¹⁴ to 3.1×10¹⁵ vp/ml of the AEX media and about 1% to 60% full capsid particles. In some embodiments of the methods of the present disclosure, the viral capsid preparation comprises about 6.7×10¹⁴ to 3.1×10¹⁵ vp/ml of the AEX media and about 4% to 11% full capsid particles. In some embodiments of the methods of the present disclosure, the viral capsid preparation comprises about 6.7×10¹⁴ to 3.1×10¹⁵ vp/ml of the AEX media and about 4% full capsid particles. In some embodiments of the methods of the present disclosure, the viral capsid preparation comprises about 6.7×10¹⁴ to 3.1×10¹⁵ vp/ml of the AEX media and about 11% full capsid particles.

In some embodiments of the methods of the present disclosure, the volume of the viral capsid preparation applied to or contacted with the AEX medium comprises between 6.5×10¹⁴ to 5.5×10¹⁵ vp/ml of the AEX media and about 1% to about 60% full capsid particles. In some embodiments of the methods of the present disclosure, the volume of the viral capsid preparation applied to or contacted with the AEX medium comprises between 6.5×10¹⁴ to 5.5×10¹⁵ vp/ml of the AEX media and about 4% full capsid particles. In some embodiments of the methods of the present disclosure, the volume of the viral capsid preparation applied to or contacted with the AEX medium comprises between 6.5×10¹⁴ to 5.5×10¹⁵ vp/ml of the AEX media and about 11% full capsid particles.

In some embodiments of the methods of the present disclosure, the volume of the viral capsid preparation applied to or contacted with the AEX medium comprises between 6.5×10¹⁴ to 5.5×10¹⁵ vp/ml of the AEX media and about 1% to about 60% full capsid particles. In some embodiments of the methods of the present disclosure, the volume of the viral capsid preparation applied to or contacted with the AEX medium comprises between 6.5×10¹⁴ to 5.5×10¹⁵ vp/ml of the AEX media and about 4% to about 11% full capsid particles. In some embodiments of the methods of the present disclosure, the volume of the viral capsid preparation applied to or contacted with the AEX medium comprises between 6.5×10¹⁴ to 5.5×10¹⁵ vp/ml of the AEX media and about 4% full capsid particles.

In some embodiments of the methods of the present disclosure, the volume of the viral capsid preparation applied to or contacted with the AEX medium comprises between 5.5×10¹⁴ to 3.5×10¹⁵ vp/ml of the AEX media and about 1% to about 60% full capsid particles. In some embodiments of the methods of the present disclosure, the volume of the viral capsid preparation applied to or contacted with the AEX medium comprises between 5.5×10¹⁴ to 3.5×10¹⁵ vp/ml of the AEX media and about 4% to about 11% full capsid particles. In some embodiments of the methods of the present disclosure, the volume of the viral capsid preparation applied to or contacted with the AEX medium comprises between 5.5×10¹⁴ to 3.5×10¹⁵ vp/ml of the AEX media and about 4% full capsid particles. In some embodiments of the methods of the present disclosure, the volume of the viral capsid preparation applied to or contacted with the AEX medium comprises between 5.5×10¹⁴ to 3.5×10¹⁵ vp/ml of the AEX media and about 11% full capsid particles.

In some embodiments of the methods of the present disclosure, the volume of the viral capsid preparation applied to or contacted with the AEX medium comprises between 7×10¹⁴ to 3×10¹⁵ vp/ml of the AEX media and about 1% to 60% full capsid particles. In some embodiments of the methods of the present disclosure, the volume of the viral capsid preparation applied to or contacted with the AEX medium comprises between 7×10¹⁴ to 3×10¹⁵ vp/ml of the AEX media and about 4% to about 11% full capsid particles. In some embodiments of the methods of the present disclosure, the volume of the viral capsid preparation applied to or contacted with the AEX medium comprises between 7×10¹⁴ to 3×10¹⁵ vp/ml of the AEX media and about 4% full capsid particles. In some embodiments of the methods of the present disclosure, the volume of the viral capsid preparation applied to or contacted with the AEX medium comprises between 7×10¹⁴ to 3×10¹⁵ vp/ml of the AEX media and about 11% full capsid particles.

In some embodiments of the methods of the present disclosure, the volume of the viral capsid preparation applied to or contacted with the AEX medium is between 100 to 1000 (e.g., 100 to 200, 200 to 300, 300 to 400, 400 to 500, 500 to 600, 600 to 700, 700 to 800, 800 to 900 or 900 to 1000, 200 to 900, 300 to 800, 400 to 700, 450 to 650, 500 to 600, and all integers including and in between 100 to 1000) ml per ml of the AEX medium. In some embodiments of the methods of the present disclosure, the volume of the viral capsid preparation applied to or contacted with the AEX medium is between 200 to 800 (e.g., 200 to 300, 300 to 400, 400 to 500, 500 to 600, 600 to 700 or 700 to 800, 300 to 700, 400 to 600, 450 to 550, and all integers including and in between 200 to 800) ml per ml of the AEX medium.

In some embodiments of the methods of the present disclosure, the flowthrough generated by passing the viral capsid preparation through the at least one AEX medium comprises at least 50% (e.g., at least 50%, at least 55%, at least 60%, at least 65%, at least 70%, at least 75%, at least 80%, at least 85%, at least 90%, at least 95% or 100%) of the empty capsids present in the volume of the viral capsid preparation. In some embodiments of the methods of the present disclosure, the flowthrough generated by passing the viral capsid preparation through the at least one AEX medium comprises at least 90% (e.g., at least 90%, at least 92%, at least 93%, at least 94%, at least 95%, at least 96%, at least 97%, at least 98%, at least 99% or 100%) of the empty capsids present in the volume of the viral capsid preparation. In some embodiments of the methods of the present disclosure, the flowthrough generated by passing the viral capsid preparation through the at least one AEX medium comprises 99% of the empty capsids present in the volume of the viral capsid preparation.

In some embodiments of the methods of the present disclosure, the flowthrough generated by passing the viral capsid preparation through the at least one AEX medium comprises less than or equal to 5% (e.g., 5%, less than 5%, less than 4%, less than 3%, less than 2%, less than 1% or 0%) of the full capsid particles present in the volume of the viral capsid preparation. In some embodiments of the methods of the present disclosure, the flowthrough generated by passing the viral capsid preparation through the at least one AEX medium comprises less than 5% (e.g., less than 5%, less than 4%, less than 3%, less than 2%, less than 1% or 0%) of the full capsid particles present in the volume of the viral capsid preparation. In some embodiments of the methods of the present disclosure, the flowthrough generated by passing the viral capsid preparation through the at least one AEX medium comprises 5% of the full capsid particles present in the volume of the viral capsid preparation.

In some embodiments of the methods of the present disclosure, the flowthrough comprises 40% to 100% (e.g., 40% to 45%. 45% to 50%, 50% to 55%, 55% to 60%, 60% to 65%, 65% to 70%, 70% to 75%, 75% to 80%, 80% to 85%, 85% to 90%, 90% to 95% or 95% to 100%, 45% to 95%, 50% to 90%, 55% to 85%, 60% to 80%, 65% to 75% and all percentage amounts in including and between 40% to 100%) of the empty capsids present in the viral capsid preparation. In some embodiments of the methods of the present disclosure, the flowthrough comprises at least 50% (e.g., at least 50%, at least 60%, at least 70%, at least 80%, at least 90%, at least 95%, at least 97%, at least 99% or 100%) of the empty capsids present in the viral capsid preparation. In some embodiments of the methods of the present disclosure, the flowthrough comprises 100% of the empty capsids present in the viral capsid preparation.

In some embodiments of the methods of the present disclosure, the flowthrough generated by passing the viral capsid preparation through the at least one AEX medium comprises less than or equal to 5% (e.g., 5%, less than 5%, less than 4%, less than 3%, less than 2%, less than 1% or 0%, and all % amounts including and in between 5% to 0%) of the full capsid particles present in the viral capsid preparation. In some embodiments of the methods of the present disclosure, the flowthrough generated by passing the viral capsid preparation through the at least one AEX medium comprises less than 5% (e.g., less than 5%, less than 4%, less than 3%, less than 2%, less than 1% or 0%) of the full capsid particles present in the viral capsid preparation. In some embodiments of the methods of the present disclosure, the flowthrough generated by passing the viral capsid preparation through the at least one AEX medium comprises 5% of the full capsid particles present in the viral capsid preparation.

Methods of the present disclosure typically include an elution step using an elution solution comprising a salt, wherein the concentration of the salt in that elution solution remains constant (that is, is held isocratically) or changes throughout the wash step. In some embodiments, the salts in the elution solution may comprise quaternary ammonium salt(s). In some embodiments, the concentrations of all salts in the elution solution comprising quaternary ammonium salt are held isocratically throughout the elution step. In some embodiments, the concentrations of all components in the elution solution comprising the quaternary ammonium salt are held isocratically throughout the elution step.

### Isocratic Separation

As mentioned, in many embodiments, (i) the concentration of the quaternary salt in the elution solution remains constant throughout the step during which the at least one elution solution is passed through or applied to the at least one anion exchange medium and/or (ii) the elution solution's salt composition remains constant throughout the step during which the elution solution is applied to or contacted with the at least one anion exchange medium. In some embodiments, concentrations of additional components of the at least one elution solution also remain constant throughout the step.

In some embodiments, one or both of (1) the step of applying to or contacting the second or a subsequent elution solution to the at least one anion exchange medium involves an isocratic separation. In some embodiments, the salt composition of the second or a subsequent elution solution and/or the salt composition of the second or a subsequent elution solution remains constant throughout the steps during which the solutions are passed through or applied to the anion exchange medium.

In some embodiments, first elution solution's salt composition, the second elution solution's salt composition, and any subsequent elution solution's salt composition remain constant throughout each individual wash or elution step.

### Gradient Separation

In certain embodiments, one or more elution steps in a method disclosed herein comprises using a solution whose composition varies during that step or those steps. For example, in a gradient separation step, the concentration of a salt in the elution solution may gradually and continually increase (e.g., linearly or stepwise) over time throughout the step.

In some embodiments of the methods of the present disclosure, the concentration of salt within the elution solution increases continuously over time throughout each individual elution step. In some embodiments of the methods of the present disclosure, the concentration of salt within the elution solution increases linearly over time throughout each individual elution step. In some embodiments of the methods of the present disclosure, the concentration of salt within the elution solution increases stepwise throughout each individual elution step.

### Equilibrating Solutions

Any of a variety of equilibrating solutions may be suitable for use in accordance with methods of the present disclosure. In some embodiments, the pH and ionic strength of the buffer equilibrating solution is chosen based on characteristics of the viral capsid preparation and/or the type of anion exchange medium or media being used.

In some embodiments, the equilibrating solution comprises NaCl. For example, in some embodiments, the elution solution comprises NaCl at a concentration of about 2 mM to about 200 mM (e.g., about 2 mM to about 200 mM, about 2 mM to about 175 nM, about 2 mM to about 150 mM, about 2 mM to about 125 mM, about 2 mM to about 100 mM, about 2 mM to about 90 mM, about 2 mM to about 80 mM, about 5 mM to about 70 mM, about 5 mM to about 60 mM, about 5 mM to about 50 mM, about 10 mM to about 50 mM, about 15 mM to about 50 mM, about 15 mM to about 40 mM, about 20 mM to about 40 mM, or about 20 mM to about 30 mM. For example, in some embodiments, the equilibrating solution comprises NaCl at a concentration of about 20 mM, about 25 mM, or about 30 mM. In some embodiments, the equilibrating solution comprises NaCl at a concentration of about 25 mM.

In some embodiments, the equilibrating solution comprises a divalent salt (e.g., MgCl₂). For example, in some embodiments, the equilibrating solution comprises MgCl₂ at a concentration of about 1 mM to about 10 mM (e.g., about 1 mM to about 10 mM, about 1 mM to about 9 mM, about 1 mM to about 8 mM, about 1 mM to about 7 mM, about 1 mM to about 6 mM, about 1 mM to about 5 mM, 1 mM to about 4 mM, or about 1 mM to about 3 mM). In some embodiments, the equilibrating solution comprises MgCl₂ at a concentration of about 1 mM, about 2 mM, or about 3 mM. In some embodiments, the equilibrating solution comprises MgCl₂ at a concentration of about 2 mM.

In some embodiments, the equilibrating solution further comprises bis-tris-propane (BTP). For example, in some embodiments, the equilibrating solution comprises BTP at a concentration of between 1 mM and 100 mM, (e.g., about 2 mM to about 200 mM, about 2 mM to about 175 mM, about 2 mM to about 150 mM, about 2 mM to about 125 mM, about 2 mM to about 100 mM, about 2 mM to about 90 mM, about 2 mM to about 80 mM, about 5 mM to about 70 mM, about 5 mM to about 60 mM, about 5 mM to about 50 mM, about 10 mM to about 50 mM, about 10 mM to about 40 mM, or 10 mM to about 30 mM. For example, in some embodiments, the equilibrating solution comprises BTP at a concentration of about 10 mM, about 15 mM, about 20 mM, about 25 mM, or about 30 mM. In some embodiments, the equilibrating solution comprises BTP at a concentration of about 20 mM.

In some embodiments, the equilibrating solution comprises both NaCl and a divalent salt (e.g., MgCl₂).

In some embodiments, the equilibrating solution comprises NaCl, a divalent salt (e.g., MgCl₂), and Bis-tris-propane.

In some embodiments, the equilibrating solution has a pH around a certain value or within a certain range, a pH from about 6.0 to about 10.0, e.g., from about 7.0 to about 9.5. In some embodiments, the equilibrating solution has a pH at about 6.0, about 6.5, about 7.0, about 7.5, about 8.0, about 8.5, about 9.0, about 9.5, or about 10.0. In some embodiments, the equilibrating solution has a pH of about pH 9.0.

As a non-limiting example, in some embodiments, the equilibrating solution is 20 mM BTP, 25 mM NaCl, 2 mM MgCl₂, pH 9.0.

### Elution Solutions and Fractions

Methods of the present disclosure generally comprise a step of passing through or applying to an anion exchange medium an elution solution, e.g., to elute full capsid particles in an elution fraction. In some embodiments, the elution solution comprises a quaternary ammonium salt. In some embodiments, the elution solution does not comprise a quaternary ammonium salt.

In some embodiments of the methods of the present disclosure, the eluent generated by passing the eluent solution through the at least one AEX medium comprises at least 50% (e.g., at least 50%, at least 55%, at least 60%, at least 65%, at least 70%, at least 75%, at least 80%, at least 85%, at least 90%, at least 95%, at least 97%, at least 99% or 100%) of the full capsid particles present in the volume of viral capsid preparation. In some embodiments of the methods of the present disclosure, the eluent generated by passing the eluent solution through the at least one AEX medium comprises at least 90% (e.g., at least 90%, at least 92%, at least 93%, at least 94%, at least 95%, at least 96%, at least 97%, at least 98%, at least 99% or 100%) of the full capsid particles present in the volume of viral capsid preparation. In some embodiments of the methods of the present disclosure, the eluent generated by passing the eluent solution through the at least one AEX medium comprises at least 99% of the full capsid particles present in the volume of viral capsid preparation.

In some embodiments of the methods of the present disclosure, the concentration of salt within the elution solution increases continuously over time throughout each individual elution step. In some embodiments of the methods of the present disclosure, the concentration of salt within the elution solution increases linearly over time throughout each individual elution step. In some embodiments of the methods of the present disclosure, the concentration of salt within the elution solution increases stepwise throughout each individual elution step.

In some embodiments, the methods of the present disclosure comprise passing 1 to 200 (e.g., 1 to 10, 10 to 20, 20 to 30, 30 to 40, 40 to 50, 50 to 60, 60 to 70, 70 to 80, 80 to 90, 90 to 100, 10 to 90, 20 to 80, 30 to 70, 40 to 60 or 45 to 55 and all integers including and in between 1 to 200) total column volumes of elution solution through the at least one AEX medium. In some embodiments, the methods of the present disclosure comprise passing 50 to 150 (e.g., 50 to 60, 60 to 70, 70 to 80, 80 to 90, 90 to 100, 100 to 110, 110 to 120, 120 to 130, 130 to 140, 140 to 150, 60 to 140, 70 to 130, 80 to 120, 90 to 110 and all integers including and in between 50 to 150) total column volumes of elution solution through the at least one AEX medium. In some embodiments, the methods of the present disclosure comprise passing 90 total column volumes of elution solution through the at least one AEX medium.

In some embodiments, the flowthrough comprises less than or equal to 5% of the full capsid particles present in the viral capsid preparation.

In some embodiments, the methods of the present disclosure, the eluent from the at least one AEX medium comprises at least 20% (e.g., at least 20%, at least 30%, at least 40%, at least 50%, at least 60%, at least 70%, at least 80%, at least 90%, at least 95%, at least 97%, at least 98%, at least 99% or 100%) of the full capsid particles present in the volume of viral capsid preparation. In some embodiments, the methods of the present disclosure, the eluent from the at least one AEX medium comprises 20% to 50% (e.g., 20% to 25%, 25% to 30%, 30% to 35%, 35% to 40%, 40% to 45%, 45% to 50%, 20% to 30%, 20% to 45%, 25% to 45%, 30% to 45%, 35% to 45% and all % amounts including and in between 20% to 50%) of the full capsid particles present in the volume of viral capsid preparation. In some embodiments, the methods of the present disclosure, the eluent from the at least one AEX medium comprises 50% to 100% (e.g., 50% to 60%, 60% to 70%, 70% to 80%, 80% to 90% or 90% to 100%, 60% to 90%, 50% to 70%, 60% to 80%, 70% to 90% and all % amounts including and in between 50% to 100%) of the full capsid particles present in the volume of viral capsid preparation.

In some embodiments of the methods of the present disclosure, the eluent from the at least one AEX medium comprises no more than 10% (e.g., 10% to 9%, 9% to 8%, 8% to 7%, 7% to 6%, 6% to 5%, 5% to 4%, 4% to 3%, 3% to 2%, 2% to 1%, 1% to 0%, 9% to 1%, 8% to 2%, 7% to 3%, 8% to 4%, 9% to 6% or 6% to 5%, and all % amounts including and in between 10% to 0%) of the empty capsid particles present in the volume viral capsid preparation. In some embodiments of the methods of the present disclosure, the eluent from the at least one AEX medium comprises no more than 5% (e.g., 5% to 4%, 4% to 3%, 3% to 2%, 2% to 1%, 1% to 0%, 4% to 1% or 3% to 2% and all % amounts including and in between 5% to 0%) of the empty capsid particles present in the volume viral capsid preparation. In some embodiments of the methods of the present disclosure, the eluent from the at least one AEX medium comprises no more than 1% (e.g., 1% to 0.9%, 0.9% to 0.8%, 0.8% to 0.7%, 0.7% to 0.6%, 0.6% to 0.5%, 0.5% to 0.4%, 0.4% to 0.3%, 0.3% to 0.2%, 0.2% to 0.1%, 0.1% to 0%, 0.9% to 0.1%, 0.8% to 0.2%, 0.7% to 0.3%, 0.6% to 0.4% or 0.55% to 0.45% and all % amounts including and in between 1% to 0%) of the empty capsid particles present in the volume viral capsid preparation. In some embodiments of the methods of the present disclosure, the eluent from the at least one AEX medium does not comprise any empty capsid particles.

In some embodiments, the methods of the present disclosure further comprise combining all of the eluent generated from each of the at least one AEX medium to form a combined eluent. In some embodiments, the combined eluent comprises 1 to 20 (e.g., 1 to 2, 2 to 4, 4 to 6, 6 to 8, 8 to 10, 10 to 12, 12 to 14, 14 to 16, 16 to 18, 18 to 20, 2 to 18, 4 to 16, 6 to 16, 8 to 14, 14 to 15 or 15 to 16 and all integers including and in between 1 to 20) column volumes for each of the at least one AEX media. In some embodiments, the methods of the present disclosure further comprise combining all of the eluent generated from each of the at least one AEX medium to form a combined eluent. In some embodiments, the combined eluent comprises 1 to 10 (e.g., 1 to 2, 2 to 3, 3 to 4, 4 to 5, 5 to 6, 6 to 7, 7 to 8, 8 to 9, 9 to 10, 2 to 9, 3 to 8, 4 to 7, 4 to 6 or 5 to 7, and all integers including and in between 1 to 10) column volumes for each of the at least one AEX media.

### Elution Solution Components

For example, in some embodiments, the elution solution comprises about 25 mM to about 375 mM (e.g., about 25 mM to about 50 mM, about 50 mM to about 75 mM, about 75 mM to about 100 mM, about 100 mM to about 125 mM, about 125 mM to about 150 mM, about 150 mM to about 175 mM, about 175 mM to about 200 mM, about 200 mM to about 225 mM, about 225 mM to about 250 mM, about 250 mM to about 275 mM, about 275 mM to about 300 mM, about 300 mM to about 325 mM, about 325 mM to about 375 mM, about 50 mM to 325 mM, about 50 mM to about 325 mM, about 75 mM to about 300 mM, about 100 mM to about 275 mM, about 125 mM to about 250 mM, about 150 mM to about 225 mM, about 175 mM to about 200 mM, and all integers including and in between about 25 mM to about 375 mM) NaCl. For example, in some embodiments, the elution solution comprises NaCl between about 70 mM to about 140 mM (e.g., about 70 mM to about 80 mM, about 80 mM to about 90 mM, about 90 mM to about 100 mM, about 100 mM to about 110 mM, about 110 mM to about 120 mM, about 120 mM to about 130 mM, about 130 mM to about 140 mM, about 80 mM to about 130 mM, about 90 mM to about 120 mM, and all integers including and in between 70 mM to 140 mM) NaCl.

In some embodiments of the methods of the present disclosure, the one or more of the first elution solution, the second elution solution, and any subsequent elution solution comprises a pH around a certain value or within a certain range, a pH of about 6 to about 10 (e.g., about 6 to about 7, about 7 to about 8, about 8 to about 9, about 9 to about 10, about 6 to about 8, about 7 to about 9 or about 8 to about 10, and all integers including and in between about 6 to about 10). In some embodiments, the one or more of the first elution solution, the second elution solution, and any subsequent elution solution comprises a pH of about 6.0, about 6.5, about 7.0, about 7.5, about 8.0, about 8.5, about 9.0, about 9.5, or about 10.0. In some embodiments, the one or more of the first elution solution, the second elution solution, and any subsequent elution solution comprises a pH of about pH 9.0.

In some embodiments, one or more of the first elution solution, the second elution solution, and any subsequent elution solution comprise a buffer system to maintain the pH between about 6 and about 10. Non-limiting examples of suitable buffer systems include bis-Tris Propane-based buffers, such as a 20 mM bis-Tris Propane system.

In some embodiments, one or more of the first elution solution, the second elution solution, and any subsequent elution solution comprise a stabilizer or a surfactant, e.g., a non-ionic surfactant. Non-limiting examples of suitable non-ionic surfactants include, e.g., Pluronic F-68. In some embodiments, the stabilizer or surfactant is present in a solution at a concentration of about 0.0001%, about 0.0005%, or about 0.001%. In some embodiments, the stabilizer or surfactant is present in a solution at a concentration of no greater than about 0.001%.

### Wash Solutions

In some embodiments, the methods of the present disclosure further comprise applying at least a first wash solution comprising a salt through the at least one AEX medium between each of the one or more repeats. In some embodiments, the methods of the present disclosure further comprise applying a first and a second wash solution comprising a salt through the at least one AEX medium between each of the one or more repeats. the methods of the present disclosure further comprise applying a first, a second and at least a third wash solution comprising a salt through the at least one AEX medium between each of the one or more repeats. In some embodiments, the methods of the present disclosure further comprise applying a first and a second wash solution comprising a salt through the at least one AEX medium between each of the one or more repeats. the methods of the present disclosure further comprise applying a first, a second, a third and at least a fourth wash solution comprising a salt through the at least one AEX medium between each of the one or more repeats.

Wash solutions compatible for use in the presently disclosed methods generally comprise one or more salts and optionally a buffer, such as a buffer described herein.

In some embodiments, the first wash solution does not comprise a quaternary ammonium salt. In some embodiments, the second or a subsequent wash solution does not comprise a quaternary ammonium salt. In some embodiments, one or more of the first wash solution and the second or any subsequent wash solution does not comprise a quaternary ammonium salt.

In some embodiments, the first wash solution comprises a quaternary ammonium salt. In some embodiments, the second or a subsequent wash solution comprises a quaternary ammonium salt. In some embodiments, one or more of the first wash solution and the second or any subsequent wash solution comprises a quaternary ammonium salt.

In some embodiments, the concentration of the quaternary ammonium salt in the first, second and/or any subsequent wash solution is about 30 mM to about 200 mM (e.g., about 30 mM and about 50 mM, about 50 mM and about 75 mM, about 75 mM to about 100 mM, about 100 mM and about 125 mM, about 125 mM and about 150 mM, about 150 mM and about 175 mM, about 175 mM and about 200 mM, about 50 mM to 175 mM, about 75 mM to 150 mM, or about 100 mM to about 125 mM, and all integers including and in between 30 mM to 200 mM). In some embodiments, the concentration of the quaternary ammonium salt in the first, second and/or any subsequent wash solution is at least 30 mM, at least 50 mM, at least 70 mM, at least 90 mM, at least 95 mM, at least 98 mM, at least 99 mM or at least 100 mM. In some embodiments, the concentration of the quaternary ammonium salt in the first, second and/or any subsequent wash solution is no more than 200 mM, no more than 180 mM, no more than 160 mM, no more than 150 mM, no more than 140 mM, no more than 130 mM, or no more than 120 mM.

In some embodiments, the quaternary ammonium salt is a tetraalkylammonium salt, e.g., a tetraalkylammonium chloride or a tetraalkylammonium acetate. In some embodiments, the quaternary ammonium salt is a tetraalkylammonium chloride selected from the group consisting of tetramethylammonium chloride (TMAC), tetraethylammonium chloride (TEAC), tetrapropylammonium chloride (TPAC), tetrabutylammonium chloride (TBAC), benzyltributylammonium chloride (BTBAC), or any combination(s) thereof. In some embodiments, the quaternary ammonium salt is tetraethylammonium chloride (TEAC).

In some embodiments, the quaternary ammonium salt is a tetraalkylammonium acetate selected from the group consisting of tetramethylammonium acetate, tetraethylammonium acetate (TEA-Ac), tetrapropylammonium acetate, tetrabutylammonium acetate, and any combination(s) thereof. In some embodiments, the quaternary ammonium salt is TEA-Ac. In some embodiments, the quaternary ammonium salt is choline chloride.

In some embodiments, the first wash solution comprises a divalent salt, e.g., MgCl₂. For example, in some embodiments, the first wash solution comprises about 1 mM to about 10 mM (e.g., about 1 mM to about 2 mM, about 2 mM to about 3 mM, about 3 mM to about 4 mM, about 4 mM to about 5 mM, about 5 mM to about 6 mM, about 6 mM to about 7 mM, about 7 mM to about 8 mM, about 9 mM to about 10 mM, about 2 mM to about 9 mM, about 3 mM to about 8 mM, about 4 mM to about 7 mM, about 5 mM to about 6 mM, and all integers included and in between about 1 mM to about 10 mM) of MgCl₂.

In some embodiments, the first wash solution comprises NaCl, Na₂SO₄, MgSO₄, or any combination thereof. For example, in some embodiments, the first wash solution comprises about 25 mM to about 375 mM (e.g., about 25 mM to about 50 mM, about 50 mM to about 75 mM, about 75 mM to about 100 mM, about 100 mM to about 125 mM, about 125 mM to about 150 mM, about 150 mM to about 175 mM, about 175 mM to about 200 mM, about 200 mM to about 225 mM, about 225 mM to about 250 mM, about 250 mM to about 275 mM, about 275 mM to about 300 mM, about 300 mM to about 325 mM, about 325 mM to about 375 mM, about 50 mM to 325 mM, about 50 mM to about 325 mM, about 75 mM to about 300 mM, about 100 mM to about 275 mM, about 125 mM to about 250 mM, about 150 mM to about 225 mM, about 175 mM to about 200 mM, and all integers including and in between about 25 mM to about 375 mM) of NaCl. For example, in some embodiments, the first wash solution comprises about 70 mM to about 140 mM NaCl (e.g., about 70 mM to about 80 mM, about 80 mM to about 90 mM, about 90 mM to about 100 mM, about 100 mM to about 110 mM, about 110 mM to about 120 mM, about 120 mM to about 130 mM, about 130 mM to about 140 mM, about 80 mM to about 130 mM, about 90 mM to about 120 mM or about 100 mM to about 120 mM, and all integers including and in between 70 mM to 140 mM) of NaCl.

In some embodiments, the first wash fraction comprises more than 50%, more than 60%, more than 70%, more than 75%, more than 80%, more than 85%, more than 90%, more than 95%, no more than 98%, no more than 99% or 100% of the salt present in the elution solution passed through the one or more AEX medium.

In some embodiments, the first wash fraction comprises less than about 5%, less than about 3%, less than about 2%, or less than about 1% or 0% of the full capsid particles present in the viral capsid preparation.

Second and/or Subsequent Wash Solutions

Methods of the present disclosure applying at least a first wash solution comprising a salt through the at least one AEX medium between each of the one or more repeats generally comprise a step of passing through or applying to an anion exchange medium a second or a subsequent wash solution, e.g., to obtain a second and/or a subsequent wash fraction, such as a wash fraction comprising the salt that was present in the first wash solution. In many embodiments, the second or a subsequent wash solution does not comprise a salt.

In some embodiments, the second or a subsequent wash solution comprises a divalent salt, e.g., MgCl₂. For example, in some embodiments, the a second or a subsequent wash solution comprises 1 mM to about 10 mM (e.g., about 1 mM to about 2 mM, about 2 mM to about 3 mM, about 3 mM to about 4 mM, about 4 mM to about 5 mM, about 5 mM to about 6 mM, about 6 mM to about 7 mM, about 7 mM to about 8 mM, about 9 mM to about 10 mM, about 2 mM to about 9 mM, about 3 mM to about 8 mM, about 4 mM to about 7 mM, about 5.5 mM to about 7.5 mM, and all integers including and in between about 1 mM to about 10 mM) MgCl₂.

In some embodiments, the second and/or any subsequent wash solution comprises NaCl, Na₂SO₄, MgSO₄, or any combination thereof. For example, in some embodiments, the second wash solution comprises about 25 mM to about 375 mM (e.g., about 25 mM to about 50 mM, about 50 mM to about 75 mM, about 75 mM to about 100 mM, about 100 mM to about 125 mM, about 125 mM to about 150 mM, about 150 mM to about 175 mM, about 175 mM to about 200 mM, about 200 mM to about 225 mM, about 225 mM to about 250 mM, about 250 mM to about 275 mM, about 275 mM to about 300 mM, about 300 mM to about 325 mM, about 325 mM to about 375 mM, about 50 mM to 325 mM, about 50 mM to about 325 mM, about 75 mM to about 300 mM, about 100 mM to about 275 mM, about 125 mM to about 250 mM, about 150 mM to about 225 mM, about 175 mM to about 200 mM, and all integers including and in between about 25 mM to about 375 mM) of NaCl. For example, in some embodiments, the second and/or a subsequent wash solution comprises about 70 mM to about 140 mM NaCl (e.g., about 70 mM to about 80 mM, about 80 mM to about 90 mM, about 90 mM to about 100 mM, about 100 mM to about 110 mM, about 110 mM to about 120 mM, about 120 mM to about 130 mM, about 130 mM to about 140 mM, about 80 mM to about 130 mM, about 90 mM to about 120 mM or about 100 mM to about 120 mM, and all integers including and in between 70 mM to 140 mM) of NaCl.

In some embodiments, the second and/or a subsequent wash solution elutes at least 30%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99% or 100% of the salt present in the previous wash solution passed through the one or more AEX medium.

In some embodiments, the second and/or a subsequent wash fraction comprises less than about 5%, less than about 3%, less than about 2%, less than about 1% or 0% of the full capsid particles present in the viral capsid preparation.

In some embodiments of the methods of the present disclosure, the salt composition of the at least first wash solution and/or the second or any subsequent wash solution, are remaining constant throughout each individual wash. In some embodiments of the methods of the present disclosure, the salt composition of the at least first wash solution and/or the second or any subsequent wash solution, are remaining changes throughout each individual wash.

In some embodiments of the methods of the present disclosure, the salt composition of the at least first wash solution increases continuously throughout each individual wash. In some embodiments of the methods of the present disclosure, the salt composition of the at least first wash solution increases linearly throughout each individual wash. In some embodiments of the methods of the present disclosure, the salt composition of the at least first wash solution increases stepwise throughout each individual wash. In some embodiments of the methods of the present disclosure, the second or any subsequent wash solution increases continuously throughout each individual wash. In some embodiments of the methods of the present disclosure, the second or any subsequent wash solution increases linearly throughout each individual wash. In some embodiments of the methods of the present disclosure, the second or any subsequent wash solution increases stepwise throughout each individual wash.

In some embodiments, one or more of the first wash solution, the second wash solution, and any subsequent wash solution comprises a stabilizer or a surfactant, e.g., a non-ionic surfactant. Non-limiting examples of suitable non-ionic surfactants include, e.g., Pluronic F-68. In some embodiments, the stabilizer or surfactant is present in a solution at a concentration of about 0.0001%, about 0.0005%, or about 0.001%. In some embodiments, the stabilizer or surfactant is present in a solution at a concentration of no greater than about 0.001%.

Assessment of Viral Capsid Preparations and/or Fractions

In certain embodiments, the viral capsid preparation and/or the elution fraction, or a sample thereof, is assessed. The first, second or any subsequent wash fractions may or may not be collected. In some embodiments, at least a sample of one or more wash fractions are collected and assessed, e.g., for quality control purposes.

### Full and Empty Capsid Particles

In some embodiments, the presence and/or amount(s) of full and/or empty capsid particles in the viral capsid preparation and/or one or more eluents (or elution fractions) is assessed. Various methods are known in the art to determine or quantify the presence of full or empty capsids; many of these methods can also be used to determine amounts, e.g., relative amounts of full and empty capsids. Examples of such methods include, but are not limited to, transmission electron microscopy (TEM), sedimentation velocity-analytical ultracentrifugation (SV-AUC), charge detection mass spectrometry (CDMS), anion exchange high performance liquid chromatography (AEX-HPLC), UV spectrophotometry, and measuring capsid and genome copies by ELISA and qPCR, e.g., for quality control purposes.

In some embodiments, an ultracentrifugation method is used to assess a viral capsid preparation and/or a fraction (e.g., an elution fraction). For example, SV-AUC is a solution-state method that measures the rate of sedimentation of molecules when subject to high spinning speed that applies a centrifugal force. The sedimentation rate, measured by sedimentation coefficient s, is related to the buoyant mass, density, specific volume, and the friction force of the molecule in its formulation matrix. The s-value, when normalized to standard solution conditions of water at 20° C. (standard temperature and pressure) is known as the s_{20, w} value, a fundamental molecular parameter that defines the mass and shape as well as the conformation of the molecule. The sedimentation coefficient distribution can be quantitated by area under peak, which is directly related to the quantity of molecules at that sedimentation coefficient. The degree of accuracy of the peak area in representing the true population depends partially on the suitability of detection system, and the number of data points gathered.

SV-AUC may be applied, for example, to separate different types of capsid particles, e.g., full capsid particles from empty capsid particles. The masses of virus particles having the same virus type and serotype may differ depending on the presence of a complete vector genome (as with full capsid particles), or presence of only part of a vector genome or complete absence of a vector genome (as with empty capsid particles). For example, empty capsid particles, having less DNA than full capsids, would be lighter than full capsids and would sediment more slowly than would full capsids. Thus, the s-value in an SV-AUC method would reflect the size of DNA packaged within a viral capsid particle.

Additionally, some impurities in a sample may absorb light at a wavelength of 230 nm; these contaminants are typically more numerous than those contaminants that absorbed at 280 nm. Thus, the A260/A230 ratio may provide some indication of purity of a sample. In some embodiments, the A230 and/or A260/A230 value is assessed.

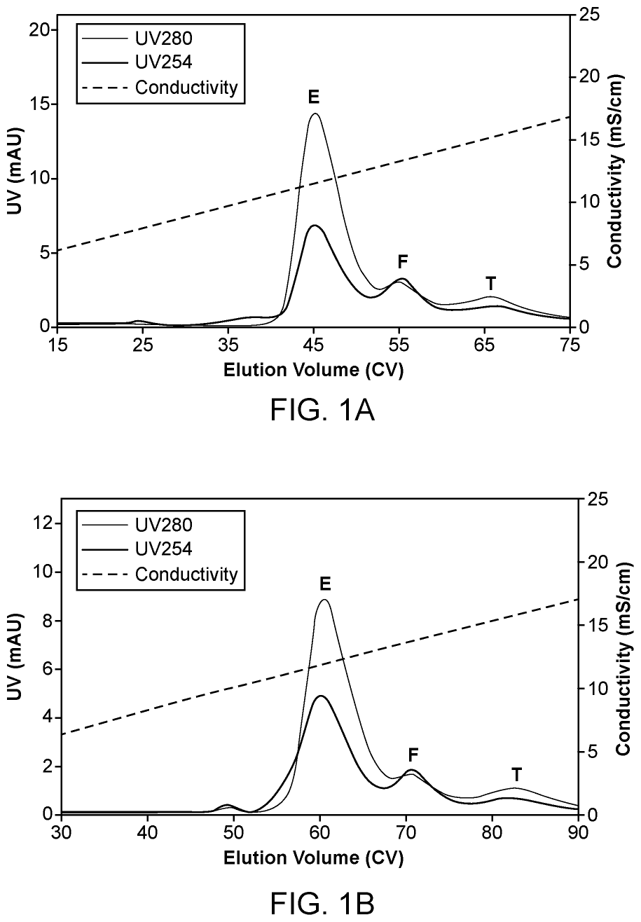

For example, in some embodiments, empty capsid particles are preferentially released from the AEX medium before full capsid particles are released. In these embodiments, the A254/A280 or A260/A280 ratio of one fraction (in which empty capsid particles are preferentially released) will be less than that of the subsequent fraction (in which full capsid particles are preferentially released) (e.g., as shown in FIG. 1). In some embodiments, the A254/A280 or A260/A280 ratio of one or more fractions are directly proportional to the amount of full capsid particles in the flow through or the eluent.

The ordering of preferential release may be associated with one or more of various aspects such as, for example (but not limited to) a) the characteristics of the nucleic acid payload, b) capsid serotype, c) viral capsid (e.g., rAAV) preparation conditions, and d) characteristics of the AEX medium.

### Other Assessments

In some embodiments, a fraction or a sample thereof is assessed to determine the presence or amount of an analyte, e.g., a component of a wash solution. For example, in some embodiments, a sample of the second wash fraction is assessed to determine the presence or amount of quaternary ammonium salt in the sample. Methods of detecting or quantitating quaternary ammonium salts are known in the art and include, e.g., liquid chromatography-mass spectrometry (LC-MS) and reversed-phase high-performance liquid chromatography (RP-HPLC).

### Anion-Exchange Media

The methods of the present disclosure are not limited to any particular column architecture or dimensions, type of separation medium, or type of separation chemistry. For example, in some embodiments, the anionic exchange medium is a weak ionic exchanger. In some embodiments, the anionic exchange medium is a strong ionic exchanger.

In some embodiments, the anion exchange (AEX) medium is in the form of a packed bed. In some embodiments, the anion exchange medium is a chromatographic monolithic column. As but one example, a CIMmultus® monolithic column (e.g., CIMmultus® monolithic QA column) may be used. In some embodiments, the monolithic column is any one of a CIMmultusC® QA column, CIMac® QA column, NuviaQ® column, POROS® HQ column, Eshmuno® Q column, POROS® XQ column, FractoGel® TMAE column and CaptoQ® column. In some embodiments, the monolithic column is a CIMmultus® QA column or a CIMac QA® column.

### Viral Capsid Preparations

Methods of the present disclosure are useful for separating full capsid particles and empty capsid particles in a viral capsid preparation, e.g., a viral capsid preparation that results from a process intended to generate a recombinant virus particle comprising a heterologous nucleic acid. Typically, the full and empty capsid particles in a given viral capsid preparation are capsid particles of the same virus and same serotype. In some embodiments, the capsid is from an AAV capsid of serotype 1, 2, 3, 4, 5, 6, 7, 8, 9, 10, 11, 12, rh10, or hu37 or a variant thereof. In some embodiments, the capsid is from an AAV capsid of serotype 8 or 9 or a variant thereof. In some embodiments, the capsid is from an AAV capsid of serotype 8 or a variant thereof. In some embodiments, the capsid is from an AAV capsid of serotype 9 or a variant thereof. In some embodiments, the capsid is from an AAV capsid of serotype rh10 or hu37 or a variant thereof. In some embodiments, the capsid is from an AAV capsid of serotype rh10 or a variant thereof. In some embodiments, the capsid is from an AAV capsid of serotype hu37 or a variant thereof.

### EXAMPLES

### Materials and Methods

AAV material comprising empty and full capsids of a recombinant AAV (rAAV) capsids was obtained from a pilot scale 250 L bioreactor operation as an example AAV load material. The clarified harvest was affinity captured and affinity eluate samples were retained for lab scale AEX runs. 1.5 ml of affinity eluate was diluted 8-fold (to 15 ml) with 20 mM Bis-tris-propane (BTP) adjusted to pH 9.0 to reduce the load conductivity to approximately 4 mS/cm. Then 10 ml of the diluted affinity eluate was loaded on AEX media (in this case, CIMac-QA (0.1 mL)) as a scale-down model for CIMmultus-QA (1 mL) pre-equilibrated with pH-matched equilibration buffer (20 mM BTP, 25 mM NaCl, 2 mM MgCl₂, pH 9.0). For elution with NaCl, a linear gradient from about 25 mM to about 182.5 mM NaCl, generated over 90 column volumes (CVs) was applied. The eluate was collected in fractions and fractions corresponding to “Empty” and “Full” peaks were pooled accordingly.

### Example 1: AEX Scale-down Model for Dynamic Binding Capacity

Objective: CIMmultus-QA column can be used in AEX-WPC operation as it was found to exhibit superior empty full rAAV separation compared to other AEX stationary phases evaluated. However, given the minimum 1 mL column volume (CV) of CIMmultus-QA, the column overloading requires prohibitively large amounts of rAAV material. To circumvent this obstacle, the study described herein determined the feasibility of using a smaller AEX column, named CIMac-QA, with 0.1 ml column volume, for determining the rAAV viral particle overloading range to integrate weak partition chromatography methodology into AEX processes (AEX-WPC).

Results: Both CIMac-QA and CIMmultus-QA are manufactured by Sartorius AG, with monolith-based stationary phase and quaternized amine ligand. To demonstrate the feasibility of CIMac-QA (0.1 ml) as a scale-down model for CIMmultus-QA (1 ml), normal batch loading runs (column underloading condition) were performed on both columns using a rAAV AEX load material comprising empty and full capsid particles, with 4% full rAAV. The results are shown in FIG. 1A and FIG. 1B. FIG. 1A and FIG. 1B show that the AEX elution chromatogram was qualitatively equivalent between CIMac-QA and CiMmultus-QA runs, with empty rAAV peak (denoted as “E”), full rAAV peak (denoted as “F”), and third peak (denoted as “T”, known to contain damaged viral particles assessed by transmission electron microscopy) sequentially resolved with increasing salt concentration. Table 1 shows that the empty and full rAAV peak conductivity, as well as the A254/A280 area ratio, were similar between CIMac-QA and CIMmultus-QA runs.

Conclusion: Based on the results of the study described herein, the 0.1 ml CIMac-QA column was demonstrated as a suitable scale-down model for 1 ml CIMmultus-QA column for AEX-WPC development.

TABLE 1
CIMac-QA as Scale-down Model for CIMmultus-QA

| | | | CIMmultus- | CIMac-QA |
|---|---|---|---|---|
| Run Name and Chromatogram Attributes | | | QA (1 ml) | (0.1 ml) |
| Normal Batch | Empty rAAV | A280 Maxima Conductivity | 11.83 | 12.05 |
| Loading Run ^{a} | Peak | (mS/cm) | | |
| | | A254/A280 | 0.48 | 0.60 |
| | | Area Ratio | | |
| | Full rAAV | A280 Maxima Conductivity | 13.62 | 14.13 |
| | Peak | (mS/cm) | | |
| | | A254/A280 | 0.97 | 1.13 |
| | | Area Ratio | | |

^{a} Load material contains 4% Full rAAV by AUC assay

### Example 2: AEX Breakthrough Curves Analysis

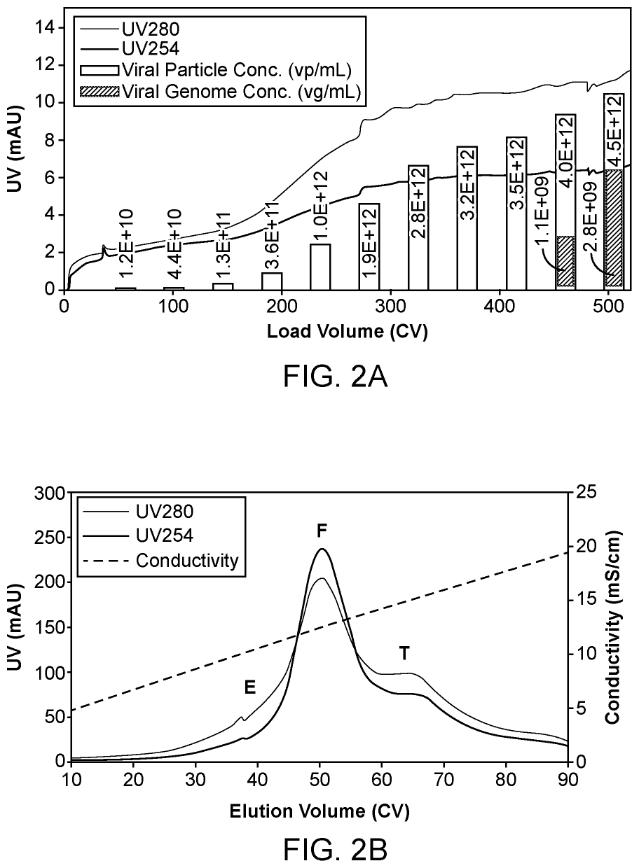

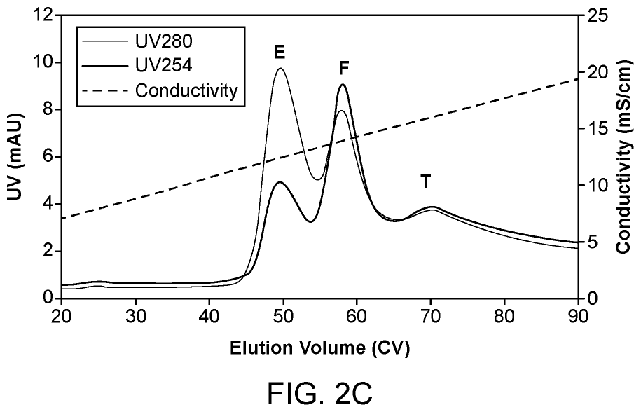

The study described herein determined the overloading range in AEX-WPC operation by measuring the dynamic binding capacity (DBC) for an rAAV AEX load material on 1 ml CIMmultus-QA column using a second lot of AEX load material which had 11% full rAAV. The fractions under the UV breakthrough curves were quantified for both viral particle concertation (vp/ml) and viral genome concentration (vg/ml). The results are shown in FIGS. 2A-2C. FIG. 2A depicts the viral particle concentration (shown as open bars) breakthrough ahead of viral genome concentration (shown as hatched bars) in the loading phase, suggesting that empty rAAVs breakthrough earlier than full rAAVs on AEX column, demonstrating WPC behavior. As a result, in the elution phase, the empty rAAV peak (denoted as “E”) (FIG. 2B) was significantly diminished when compared to that of normal batch loading run (FIG. 2C), corroborating that the empty rAAVs were indeed reduced during AEX column overloading. Based on the results of the study described herein, it was concluded that to ensure minimal full rAAV flow through during overloading, the high loading end of AEX-WPC operation should not exceed the breakthrough position of viral genome concentration in the DBC run.

### Example 3: Multi-Column Chromatography Implementation

Objective: All the studies described herein integrated the multi-column chromatography (MCC) that allows for parallel column processing capability, with AEX-WPC operation to generate AEX-WPC-MCC method, to reduce the elongated AEX processing time due to column overloading.

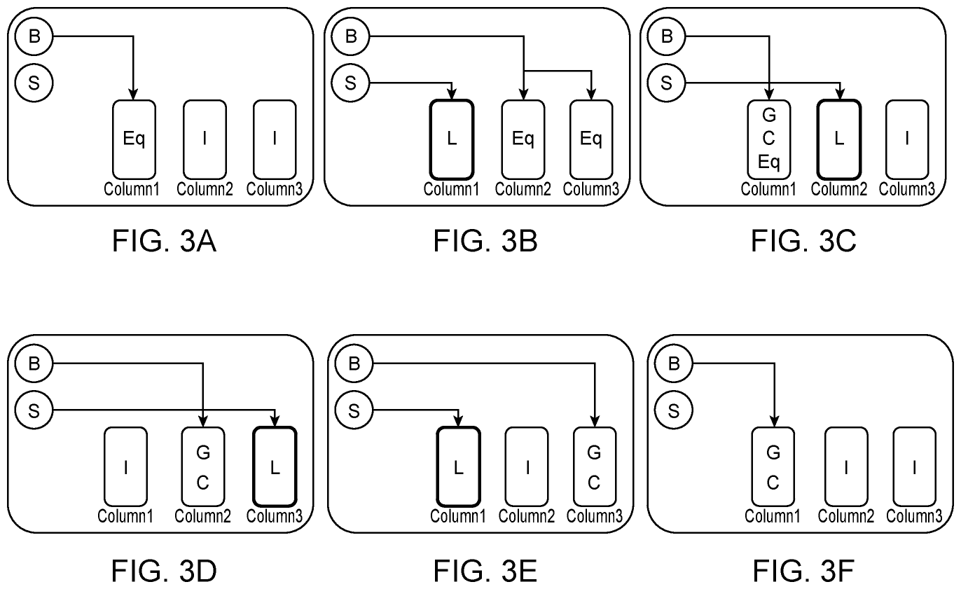

Study Design: The studies described herein used the commercially available instrument AKTA PCC75, that enables parallel multi-column processing, to execute AEX-WPC-MCC runs. One 3-column MCC loop (containing 4 AEX runs) is illustrated by 6 schematic diagrams in FIGS. 3A-3F, with each diagram depicting a representative stage in the MCC loop. For a single column, the process was started with equilibration phase (denoted as “Eq”), and then followed by loading phase (denoted as “L”) where the shaded column represents the saturation by full rAAV. Post column loading, the full rAAVs were eluted from AEX column by a salt linear gradient elution phase (denoted as “G”) and the fractions under the full rAAV peak were pooled. The eluted column then entered the cleaning phase (denoted as “C”) before it is cycled to next equilibration phase. The AEX-WPC-MCC method developed in this study did not contain the column-interconnecting phase, which is the phase when the volume of the rAAV load material passed between any two columns of the multiple-AEX column system. For column-interconnecting phase to happen, each column of the multiple AEX columns as depicted in FIGS. 3A-3F, has to be linked to the next AEX column, such that the same volume of rAAV load material is passed through all of the multiple AEX columns. Since there was no full rAAV breakthrough detected at the end of each column loading in the study described herein, there was no need to interconnect the columns and therefore, for the system to have a column interconnecting phase.

### Example 4: AEX Load Full AAV % Impact to AEX-WPC-MCC

Objective: The full rAAV % of the AEX load material could alter the relative breakthrough position between viral particle concentration and viral genome concentration, thus impacting the available loading range in AEX-WPC-MCC operation. In the study described herein, a robust loading range (low and high end loading amount of viral capsid preparation/loading material) was identified, by performing DBC runs on 0.1 ml CIMac-QA column (a scale-down model used to conserve AEX load material) using both 4% and 11% full rAAV AEX load material of an exemplary rAAV.

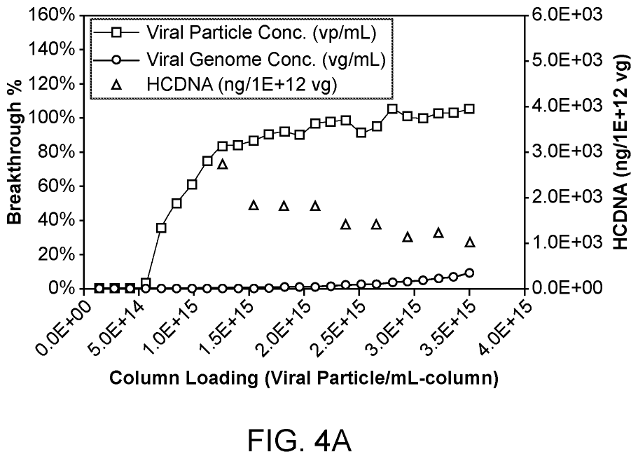

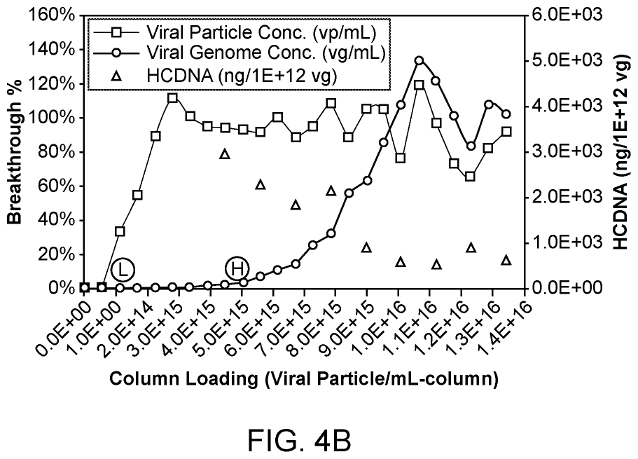

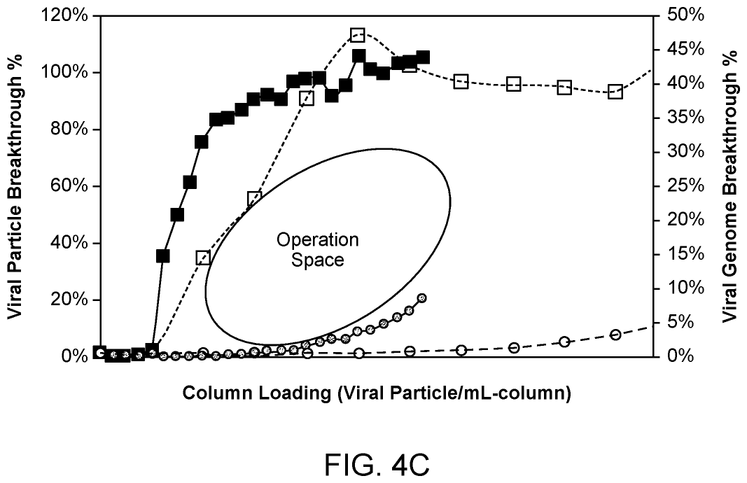

Results: The results are described in FIGS. 4A-4B and Table 2. In FIGS. 4A and 4B both viral particle breakthrough curve and viral genome breakthrough curve were plotted as a function of viral particle loading per column volume (vg/ml-column) for both 11% full rAAV material and 4% full rAAV material, respectively. Due to material limitation, the maximum DBC loading achieved for 11% full rAAV material was around 3.5×10¹⁵ vp/ml-column, as compared to maximum about 1.4×10¹⁶ vp/ml-column loading achieved for 4% full rAAV material. The detailed loading range comparation are shown in Table 2, where the 4% full rAAV material achieved column loading at 6.7×10¹⁴ vp/ml-column and 5.3×10¹⁵ vp/ml-column at 5% viral particle concentration breakthrough and 5% viral genome concentration breakthrough, respectively. In contrast, the 11% full rAAV material achieved column loading at 5.8×10¹⁴ vp/ml-column and 3.1×10¹⁵ vp/ml-column at 5% viral particle concentration breakthrough and 5% viral genome concentration breakthrough, respectively. As a result, the differential viral particle loading range for 4% full rAAV material (4.6×10¹⁵ vp/ml-column) was shown to be 1.8-fold of that for 11% full AAV material (2.6×10¹⁵ vp/ml-column). It was concluded that the overlapping range of 6.7×10¹⁴ to 3.1×10¹⁵ vp/ml-column bracketed by 4% full rAAV load material and 11% full rAAV load material served as a robust loading space for AEX-WPC-MCC operation, provided that the full rAAV % of the example AEX load material ranged from 4 to 11%.

TABLE 2
AEX Dynamic Binding Capacity Using CIMac-QA (0.1 ml)

| | | | Differential Viral | Fold of Differential |
|---|---|---|---|---|
| Load Full | Events in Dynamic | Viral Particle | Particle Loading | Viral Particle Loading |
| rAAV % | Binding Capacity | Loading (vp/ml- | Range (vp/ml- | (4% Material divided |
| ^{a} | Run | column) | column) | by 11% Material) |
| 11% | 5% Breakthrough of | 5.8 × 10¹⁴ | 2.6 × 10¹⁵ | 1.8 |
| | Viral Particle | | | |
| | Concentration | | | |
| | 5% Breakthrough of | 3.1 × 10¹⁵ | | |
| | Viral Genome | | | |
| | Concentration | | | |
| 4% | 5% Breakthrough of | 6.7 × 10¹⁴ | 4.6 × 10¹⁵ | |
| | Viral Particle | | | |
| | Concentration | | | |
| | 5% Breakthrough of | 5.3 × 10¹⁵ | | |
| | Viral Genome | | | |
| | Concentration | | | |

^{a} Full-AAV % assessed by AUC assay

In addition, FIGS. 4A-4B show that the flow through host cell DNA (HCDNA) concentration of ng/1×10¹² vg (shown as open triangles) observed in the loading phase, decreased with increase of viral particle loading for both 11% and 4% full rAAV load material. Based on the viral particle and viral genome breakthrough plotted with respect to the viral particle load of the loading material, the operation space of the AEX-WPC-MCC method for separating/enriching full rAAVs from a capsid preparation/loading material in terms of the viral load of the loading material, was defined (FIG. 4C).

Conclusion: It was determined that with properly defined loading range, AEX-WPC-MCC method could reduce HCDNA impurity from the product stream as well. Given that the detectable HCDNA at this stage of purification (post nuclease digestion step) are predominantly encapsidated and manifested as intermediate rAAV in analytical ultracentrifuge (AUC) analysis, it was concluded that intermediate rAAV could also be reduced to certain extent in AEX-WPC-MCC operation.

### Example 5: Evaluation of AEX-WPC-MCC Loading Range

Objective: In the study described herein, the process performance and product quality of AEX-WPC-MCC operation in the proposed loading range (6.7×10¹⁴ to 3.1×10¹⁵ vp/ml-column) was assessed, by performing a low-end loading run (7.0×10¹⁴ vp/ml-column, denoted by the encircled “L” in FIG. 4B) and high-end loading run (2.8×10¹⁵ vp/ml-column, denoted by the encircled “H” in FIG. 4B) using an AAV serotype 8 AEX load material with 11% full rAAV.

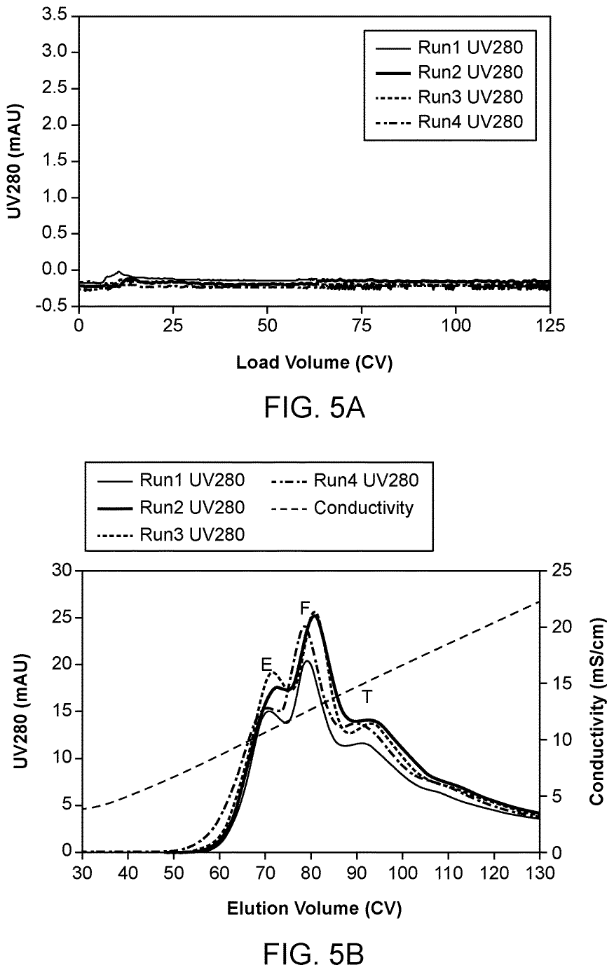

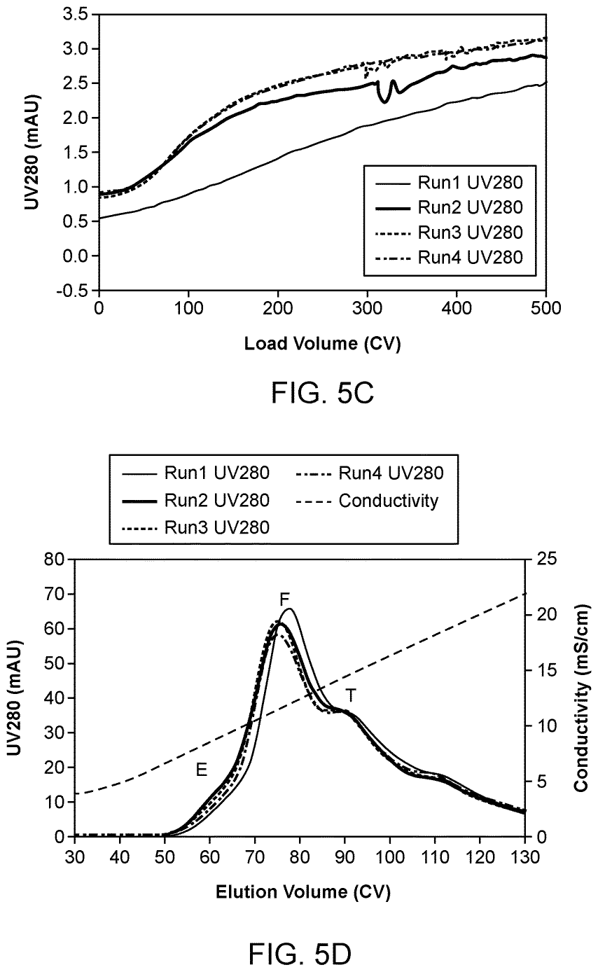

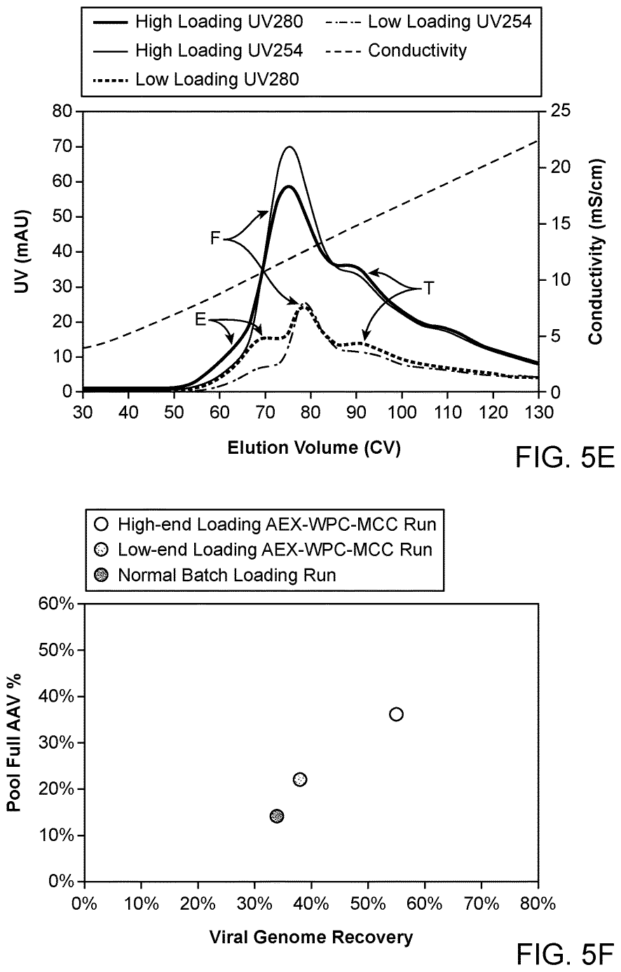

Results: FIGS. 5A-5E and Table 3 show the results of the study described herein. In the low-end loading AEX-WPC-MCC run, although there was no detectable UV280 breakthrough during loading (FIG. 5A), the gradient elution profile (FIG. 5B) showed noticeable enrichment of full rAAV peak comparing to that of normal batch loading run in FIG. 2C. In contrast, the high-end loading AEX-WPC-MCC run resulted in UV280 breakthrough in loading profile (FIG. 5C) and empty peak diminishment in gradient elution profile (FIG. 5D), resembling that of the DBC run in FIG. 2B. When gradient elution profiles of both low-end loading run and high-end loading run (Run4 from each group) were overlayed, in FIG. 5E, it showed that the full rAAV peak was further enriched in high-end loading run.

Furthermore, the fractions under full rAAV peak (8 CV flanking the apex) of both low-end and high-end loading runs were collected as AEX pools and their viral genome recovery and pool full rAAV % were determined. The results described in Table 3 showed that the viral genome recoveries were 34%, 38%, and 55% and pool full rAAV % were 14%, 22%, and 36% for normal batch loading run, low-end loading AEX-WPC-MCC run, and high-end loading AEX-WPC-MCC runs, respectively. These data were further plotted in FIG. 5F to visualize the trade-off relationship between viral genome recovery and pool full rAAV %. The AEX normal batch loading run only achieved 1% loading of the DBC cutoff (viral particle loading at 5% breakthrough of viral particle concentration using 11% full rAAV AEX load material, as shown in Table 3), comparing to 22% loading and 90% loading of the DBC cutoff for low-end and high-end loading AEX-WPC-MCC runs, respectively. Based on the results described herein, the higher pool full rAAV % of AEX-WPC-MCC runs were determined to be due to the flow through of empty rAAV during overloading, while the higher viral genome recovery of AEX-WPC-MCC runs was due to the mitigation of product loss by column overloading. Further, the results described in Table 3, showed that the AEX pool viral genome concentration (vg/ml) were also significantly increased from AEX normal batch run (1.2×10¹¹ vg/ml) to low-end loading AEX-WPC-MCC run (5.2×10¹² vg/ml) and to high-end loading AEX-WPC-MCC run (3.0×10¹³ vg/ml).

Conclusion: Based on the results described herein, the AEX-WPC-MCC operation in the loading range described herein, was determined to be effective and qualitatively high in separating empty and full AAV capsids in an AAV capsid load material. Additionally, the high viral genome concentration post AEX step is beneficial for subsequence downstream operations, including the final ultrafiltration and diafiltration (UFDF) step where recombinant AAVs (rAAVs) are concentrated, and buffer exchanged into drug substance. Assuming the rAAV drug substance viral genome concentration is in the 10¹³ vg/ml range, the 3.0×10¹³ vg/ml viral genome concentration of AEX pool could allow the UFDF step to skip the concentration phase and directly enter the buffer exchange phase, which would alleviate the current operation burden imposed by the high concentration factor (usually ˜100-fold) in the UFDF step.

TABLE 3
CIMac-QA WPC-MCC Loading Range Evaluation

| | | | Viral | Viral | | | Pool |
|---|---|---|---|---|---|---|---|
| Load | | Volumetric | Particle | Particle | | Pool | Viral |
| Full | | Loading | Loading | Loading % | Viral | Full | Genome |
| rAAV | Run | (mL/ml- | (vp/ml - | to DBC | Genome | rAAV | Conc. |
| % ^{a} | Description | column) | column) | Cutoff ^{b} | Recovery | % ^{a} | (vg/ml) |
| 11% | AEX Normal | 10 | 3.5 × | 1% | 34% ^{d} | 14% | 1.2 × |
| | Batch Loading | | 10¹³ | | | | 10^{11 d} |
| | Run | | | | | | |
| | AEX-WPC- | 200 | 7.0 × | 22% | 38% ^{c} | 22% | 5.2 × |
| | MCC Low-end | | 10¹⁴ | | | | 10^{12 c} |
| | Loading Run | | | | | | |
| | AEX-WPC- | 800 | 2.8 × | 90% | 55% ^{c} | 36% | 3.0 × |
| | MCC High- | | 10¹⁵ | | | | 10^{13 c} |
| | end Loading | | | | | | |
| | Run | | | | | | |

^{a} Full rAAV % assessed by Stunner Instrument
^{b} DBC Cutoff is the viral particle loading at 5% breakthrough of viral particle concentration using 11% full rAAV AEX load material
^{c} Viral genome recovery assessed by ddPCR assay
^{d} Viral genome recovery assessed by qPCR assay

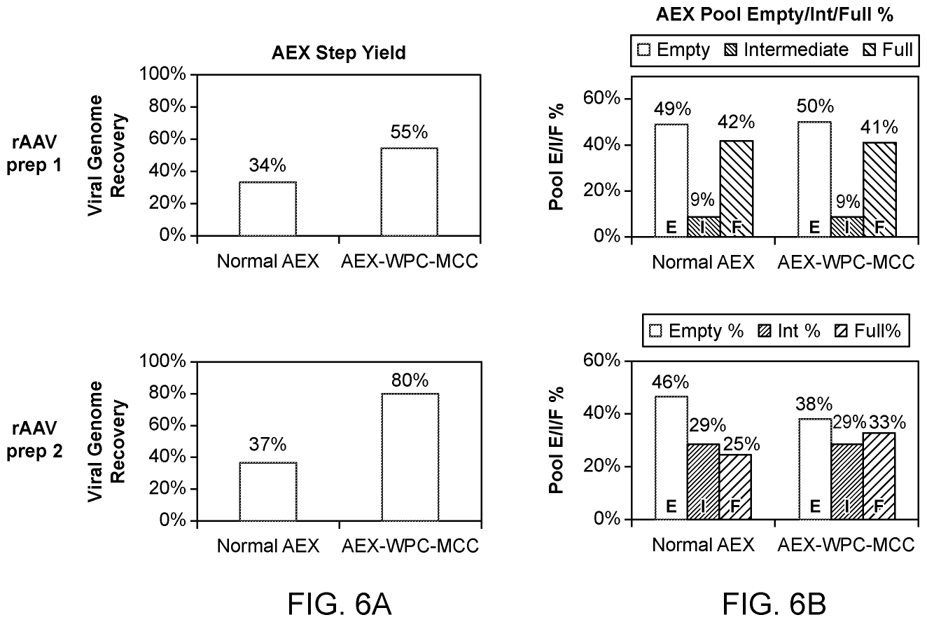

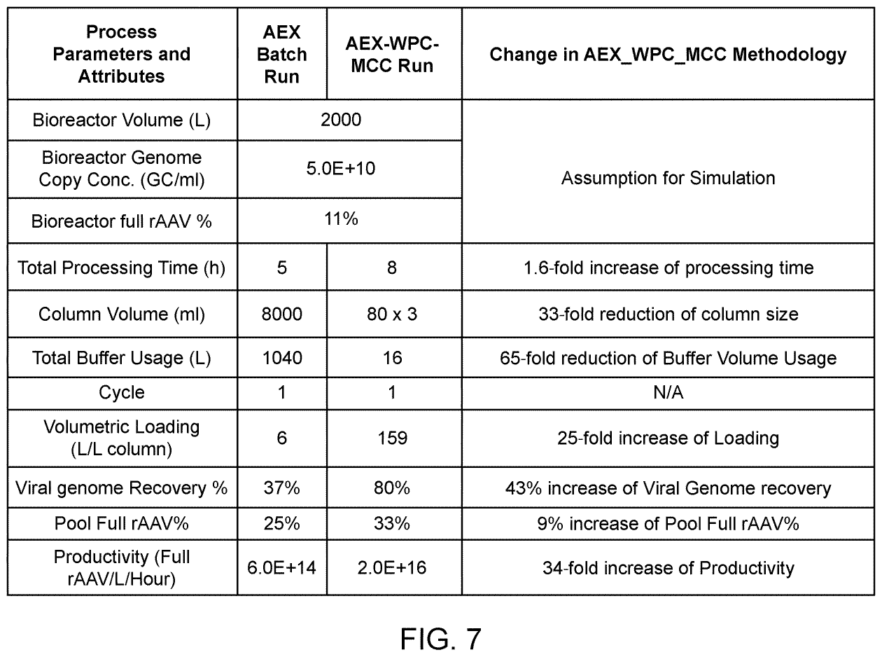

AEX-WPC-MCC overloading runs were conducted with the loading range of 6.7×10¹⁴ to 3.1×10¹⁵ vp/ml-column for two different rAAV preparation loading materials/capsid preparations (rAAV prep 1 and rAAV prep 2). Results from these overloading runs showed higher full viral particle purification (depicted by % viral genome recovery) and comparable separation of empty, intermediate, and full viral particles, as compared to normal AEX runs (FIGS. 6A-6B). The AEX-WPC-MCC overloading method with the loading ranges described herein resulted in an about 33-fold reduction in column volume, an about 65-fold reduction in buffer volume, and an about 34-fold increase in productivity (full rAAV/ml AEX column/hour) as compared to methods using AEX normal loading (FIG. 7).

### Example 6: Evaluation of Host Cell Proteins (HCP) Levels in Fractions Isolated from AEX-WPC-MCC Overloading Runs

Objective: In addition to empty and full rAAV separation, AEX chromatography is also responsible for the clearance of certain process-related impurities, such as host cell protein (HCP). In this Example, the effect of AEX overload chromatography of AAV preparations on HCP levels in isolated fractions was determined.

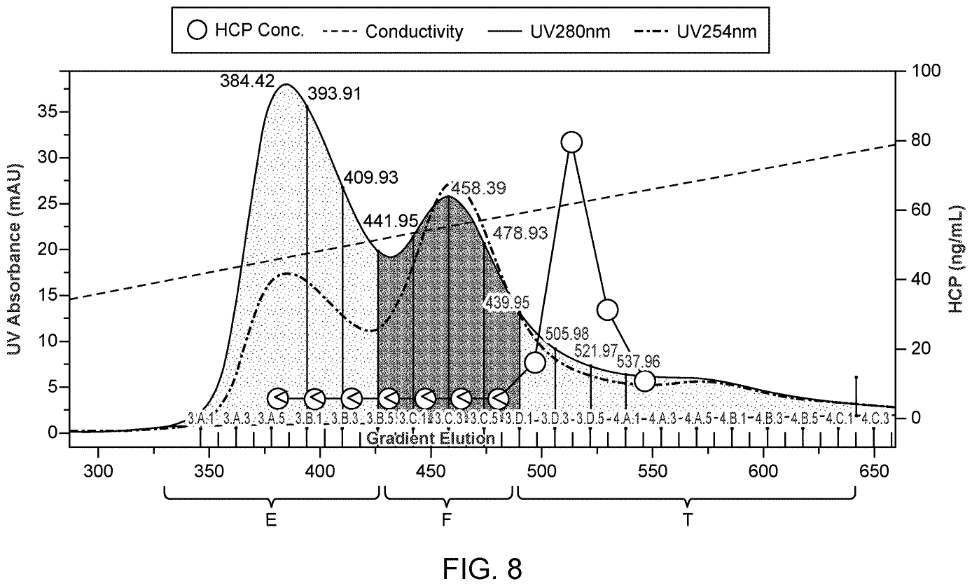

Results: In rAAV AEX salt linear gradient elution, the HCPs usually appeared in the tailing fractions of the peak of full AAV capsid particles (“full peak,” denoted “F” in FIG. 8), suggesting that these HCPs have higher net negative charges than full rAAV and will be retained on the AEX column until column cleaning. These characteristics of HCPs enable full-peak-tail-cutting as a robust method to exclude HCPs from entering the product stream in an AEX chromatography step. The AAV material was loaded onto AEX chromatography column for both a batch loading run (without loading breakthrough) and a DBC loading run (with loading breakthrough).

Table 4 below shows the genome recovery, full AAV %, and HCP levels in AAV fractions isolated using AEX overloading chromatography. Both genome recovery and full rAAV % were higher in from the AEX DBC loading run (50.5% and 20.8% respectively) as compared to AEX batch loading run (39.0% and 17.9% respectively) for AAV material. Separating empty and full AAV capsids from AAV material using AEX weak partition chromatography demonstrated that although the HCP level was detectable (12 ng/mL) in AEX load material, the HCP level was below the limit of detection (<4 ng/mL) for AEX eluates of both the AEX batch loading and the AEX DBC loading runs. In addition, the high genome titer (2.0×10¹³ GC/mL, which is in close proximity to the drug substance concentration) of AEX eluate from the AEX DBC loading run showed that similar (<4 ng/mL) HCP levels were observed in the drug substance as well.

TABLE 4
HCP concentration of AEX chromatography
using AAV load material

| | | Full rAAV | | |
|---|---|---|---|---|
| | | % by | Genome Titer | HCP |
| Sample | Genome | sv-AUC¹ | (GC/mL) by | Concentration |
| Name | Recovery | assay | qPCR assay | (ng/mL) |
| AEX Load | N/A | 9.3% | 8.3 × 10¹² | 12 |
| Material | | | | |
| AEX Batch | 39.0% | 17.9% | 6.0 × 10¹¹ | <4 |
| Loading Run | | | | |
| Eluate | | | | |
| AEX DBC² | 50.5% | 20.8% | 2.0 × 10¹³ | <4 |
| Loading Run | | | | |
| Eluate | | | | |

¹Sv-AUC: Sedimentation Velocity Analytical Ultracentrifugation Assay
²DBC: dynamic binding capacity

Conclusion: This Example demonstrated that HCPs were sufficiently removed through an AEX step despite overloading the AEX column, thereby mitigating the concern of clearance of HCPs clearance in a AEX weak partition chromatography method. AAV overloading and separation using AEX chromatography has been shown to maintain the robustness of HCP clearance (see Table 4). Furthermore, even if HCP clearance becomes a concern, the HCP reduction capability from other unit operations prior to the AEX step, such as harvest clarification, harvest tangential flow filtration, and affinity chromatography, could be leveraged to alleviate the burden of the AEX step.

Based on the results of the studies described herein, disclosed methods, which comprise integrating weak partitioning chromatography (WPC) and multiple-column chromatography (MCC) into an AEX method, achieved improved separation of empty and full rAAVs, where the empty rAAVs flow through column during column overloading. In summary, the methods disclosed herein resulted in the enhancement of both viral genome recovery and full rAAV % of AEX pool, and reduced AEX column volume and buffer usage, contributing to cost reduction of the downstream manufacturing process. Previous reports have shown that HCP could be removed by synthetic depth filters as well as by affinity chromatography, and these examples may be leveraged and adapted into an rAAV manufacturing process to achieve optimal HCP reduction through the AEX chromatography during column overloading.

We claim:

1. A method of separating empty capsid particles in a viral capsid preparation comprising empty and full capsid particles, the method comprising the steps of:
a) applying an equilibrating solution to at least one anion exchange (AEX) medium; and
b) passing a volume of the viral capsid preparation through the at least one AEX medium to generate a flowthrough comprising empty capsid particles,
wherein the volume of the viral capsid preparation comprises between 1×10¹⁴ to 5.5×10¹⁵ vp/ml of the AEX medium.

2. A method of separating full capsid particles and empty capsid particles in a viral capsid preparation comprising empty and full capsid particles, the method comprising the steps of:
a) applying an equilibrating solution to at least one anion exchange (AEX) medium;
b) passing a volume of the viral capsid preparation through the at least one AEX medium to generate a flowthrough comprising empty capsid particles; and
c) passing an elution solution comprising a salt through the at least one AEX medium to generate an eluent comprising full capsid particles,
wherein the volume of the viral capsid preparation comprises between 1×10¹⁴ to 5.5×10¹⁵ vp/ml of the AEX medium.

3. A method of increasing the percentage of full capsid particles in a viral capsid preparation comprising empty and full capsid particles, the method comprising the steps of:
a) applying an equilibrating solution to at least one anion exchange (AEX) medium;
b) passing a volume of the viral capsid preparation through the at least one AEX medium to generate a flowthrough;
c) passing an elution solution comprising a salt through the at least one AEX medium to generate an eluent; and
d) collecting the eluent from step c),
wherein a volume of the eluent of step d) comprises full capsid particles at a percentage that is higher than the percentage of full capsid particles in an equivalent volume of the viral capsid preparation, and
wherein the volume of the viral capsid preparation comprises between 1×10¹⁴ to 5.5×10¹⁵ vp/ml of the AEX medium.

4. The method of any one of claims 1-3, wherein step b) further comprises collecting at least a portion of the flowthrough and analyzing the empty and full capsid content of the collected portion of the flowthrough, wherein the passing of the volume of the viral capsid preparation through the at least one AEX-medium in step b) is stopped if full capsid particle is detected in the collected portion of the flowthrough.

5. The method of claim 4, wherein the analyzing the empty and full capsid content comprises determining the A254/A280 ratio of the collected portion of the flowthrough using UV spectrophotometry.

6. The method of any one of claims 1-5, comprising using at least two, at least three, or at least four AEX media.

7. The method of claim 6, wherein the volume of the viral capsid preparation applied in step b) is constant between each of the at least two, at least three, or at least four AEX media.

8. The method of any one of claims 1-7, comprising sequential use of at least two, at least three, or at least four AEX media.

9. The method of claim 8, wherein the sequential use comprises completing each of the steps using one AEX medium, then repeating each of the steps starting with step a) using a subsequent AEX medium.

10. The method of claim 8, wherein the sequential use comprises completing at least one of the steps with one AEX medium, then performing each of the steps starting with step a) using a subsequent AEX medium.

11. The method of any one of claims 1-10, comprising repeating each of the steps at least one, at least two, or at least three times, each time with a different AEX medium.

12. The method of claim 11, wherein the volume of the viral capsid preparation applied to the at least one AEX medium remains constant between each repetition.

13. The method of any one of claims 11-12, further comprising passing at least a first wash solution through the at least one AEX medium between each repetition.

14. An elution method comprising:
a) passing a volume of a viral capsid preparation comprising full capsid particles and empty capsid particles through at least one anion exchange (AEX) medium to generate a flowthrough comprising empty capsid particles; and
b) passing an elution solution comprising a salt through the at least one AEX medium to generate an eluent comprising full capsid particles,
wherein the volume of the viral capsid preparation comprises between 1×10¹⁴ to 5.5×10¹⁵ vp/ml of the AEX medium.

15. The method of claim 14, wherein step a) further comprises collecting at least a portion of the flowthrough and analyzing the empty and full capsid content of the collected portion of the flowthrough, wherein the passing of the volume of the viral capsid preparation through the at least one AEX-medium in step a) is stopped if full capsid particle is detected in collected portion of the flowthrough.

16. The method of claim 15, wherein the analyzing the empty and full capsid content comprises determining the A254/A280 ratio of the collected portion of the flowthrough using UV spectrophotometry.

17. The method of any one of claims 14-16, comprising conducting steps a)-b) using at least two, at least three or at least four AEX media.

18. The method of claim 17, comprising sequential use of the at least two, at least three or at least four AEX media.

19. The method of claim 18, wherein the sequential use comprises completing each of the steps using one AEX medium, then repeating each of the steps starting with step a) using a subsequent AEX medium.

20. The method of claim 18, wherein the sequential use comprise completing at least one of the steps with one AEX medium, then performing each of the steps starting with step a) using a subsequent AEX medium.

21. The method of any one of claims 14-20, comprising repeating each of the steps at least one, at least two, or at least three times, each time with a different AEX medium.

22. The method of claim 21, wherein the volume of the viral capsid preparation applied to the at least one AEX medium remains constant between each repetition.

23. The method of any one of claims 21-22, further comprises passing at least a first wash solution to the at least one AEX medium between each repetition.

24. The method of any one of claims 1-23, wherein the volume of the viral capsid preparation applied to the AEX medium comprises between 6.7×10¹⁴ to 3.1×10¹⁵ vp/ml of the AEX medium.

25. The method of any one of claims 1-23, wherein the volume of the viral capsid preparation applied to or contacted with the AEX medium comprises between 6.5×10¹⁴ to 5.5×10¹⁵ vp/ml of the AEX medium.

26. The method of any one of claims 1-23, wherein the volume of the viral capsid preparation applied to or contacted with the AEX medium comprises between 5.5×10¹⁴ to 3.5×10¹⁵ vp/ml of the AEX medium.

27. The method of any one of claims 1-23, wherein the volume of the viral capsid preparation applied to with the AEX medium comprises between 7×10¹⁴ to 3×10¹⁵ vp/ml of the AEX medium.

28. The method of any one of claims 1-27, wherein the viral capsid preparation comprises about 1% to about 60% full capsid particles.

29. The method of claim 28, wherein the viral capsid preparation comprises about 4% to about 11% full capsid particles.

30. The method of any one of claims 1-29, wherein the volume of the viral capsid preparation applied to or contacted with the AEX medium is between 100 to 1000 ml per ml of the AEX medium.

31. The method of claim 30, wherein the volume of the viral capsid preparation applied to or contacted with the AEX medium is between 200 to 800 ml per ml of the AEX medium.

32. The method of any one of claims 1-31, wherein the flowthrough comprises at least 50% of the empty capsids present in the volume of the viral capsid preparation.

33. The method of any one of claims 1-32, wherein the flowthrough comprises at least 90% of the empty capsids present in the volume of the viral capsid preparation.

34. The method of claim any one of claims 1-32, wherein the flowthrough comprises 99% of the empty capsids present in the volume of the viral capsid preparation.

35. The method of any one of claims 1-34, wherein the flowthrough comprises less than or equal to 5% of the full capsid particles present in the volume of the viral capsid preparation.

36. The method of any one of claims 2-35, wherein the eluent comprises at least 50% of the full capsid particles present in the volume of viral capsid preparation.

37. The method of any one of claims 2-35, wherein the eluent comprises at least 90% of the full capsid particles present in the volume of viral capsid preparation.

38. The method of any one of claims 2-35, wherein the eluent comprises at least 99% of the full capsid particles present in the volume of viral capsid preparation.

39. The method of any one of claims 2-38, wherein the concentration of salt within the elution solution increases continuously over time throughout each individual elution step.

40. The method of claim 38, wherein the concentration of salt within the elution solution increases linearly over time throughout each individual elution step.

41. The method of claim 38, wherein the concentration of salt within the elution solution increases stepwise throughout each individual elution step.

42. The method of any one of claims 2-41, comprising passing 1 to 200 total column volumes of elution solution through the at least one AEX medium.

43. The method of claim 42, comprising passing 50-150 total column volumes of elution solution through the at least one AEX medium.

44. The method of claim 43, comprising passing about 90 total column volumes of elution solution through the at least one AEX medium.

45. The method of any one of claims 1-44, wherein the flowthrough comprises 40% to 100% of the empty capsids present in the viral capsid preparation.

46. The method of claim 45, wherein the flowthrough comprises 100% of the empty capsids present in the viral capsid preparation.

47. The method of any one of claims 45-46, wherein the flowthrough comprises less than or equal to 5% of the full capsid particles present in the viral capsid preparation.

48. The method of any one of claims 2-47, wherein the eluent comprises at least 20% of the full capsid particles present in the viral capsid preparation.

49. The method of any one of claims 2-47, wherein the eluent comprises 20% to 50% of the full capsid particles present in the viral capsid preparation.

50. The method of any one of claims 2-47, wherein the eluent comprises 50% to 100% of the full capsid particles present in the viral capsid preparation.

51. The method of any one of claims 2-50, wherein the eluent comprises no more than 10% of the empty capsid particles present in the viral capsid preparation.

52. The method of any one of claims 2-50, wherein the eluent comprises no more than 5% of the empty capsid particles present in the viral capsid preparation.

53. The method of any one of claims 2-50, wherein the eluent comprises no more than 1% of the empty capsid particles present in the volume of the viral capsid preparation.

54. The method of any one of claims 2-50, wherein the eluent does not comprise any empty capsid particles.

55. The method of any one of claims 2-54, further comprising a step of combining all of the eluent generated from each of the at least one AEX media to form a combined eluent.

56. The method of claim 55, wherein the combined eluent comprises 1 to 20 column volumes for each of the at least one AEX media.

57. The method of claim 56, wherein the combined eluent comprises 1 to 10 column volumes for each of the at least one AEX media.

58. The method of any one of claims 2-57, wherein the elution solution comprises MgCl₂ or NaCl.

59. The method of any one of claims 2-58, wherein the elution solution comprises NaCl.

60. The method of claim 59, wherein the elution solution comprises about 25 mM to about 375 mM NaCl.

61. The method of claim 60, wherein the elution solution comprises about 90 mM to about 140 mM NaCl.

62. The method of any one of claims 58-61, wherein the elution solution comprises a pH of about 9.

63. The method of any one of claims 11, 12, 13, 21, 22, and 23, further comprising applying at least a first wash solution comprising a salt through the at least one AEX medium between each of repetition.

64. The method of claim 63, wherein the first wash solution does not comprise a quaternary ammonium salt.

65. The method of claim 63 or 64, further comprising at least a second wash solution comprising a salt through the at least one AEX medium between each repetition.

66. The method of claim 65, wherein the second or a subsequent wash solution does not comprise a quaternary ammonium salt.

67. The method of any one of claims 63-66, wherein the salt composition of the at least first wash solution and/or the second or any subsequent wash solution, remain constant throughout each individual wash.

68. The method of any one of claims 63-66, wherein the salt composition of the at least first wash solution increases continuously throughout each individual wash.

69. The method of claim 68, wherein the salt composition of the at least first wash solution increases linearly throughout each individual wash.

70. The method of claim 68, wherein the salt composition of the at least first wash solution increases stepwise throughout each individual wash.

71. The method of any one of claims 63-70, wherein the salt composition of the second or any subsequent wash solution increases continuously throughout each individual wash.

72. The method of claim 71, wherein the salt composition of the second or any subsequent wash solution increases linearly throughout each individual wash.

73. The method of claim 71, wherein the salt composition of the second or any subsequent wash solution increases stepwise throughout each individual wash.

74. The method of any one of claims 1-73, wherein the AEX medium is a monolithic column.

75. The method of claim 74, wherein the monolithic column is any one of a CIMmultus® QA column, CIMac® QA column, NuviaQ® column, POROS® HQ column, Eshmuno® Q column, POROS® XQ column, FractoGel® TMAE column and CaptoQ® column.

76. The method of claim 75, wherein the monolithic column is a CIMmultus® QA column or a CIMac QA column.

77. The method of any one of claims 1-76, wherein the capsid is from an AAV capsid of serotype 1, 2, 3, 4, 5, 6, 7, 8, 9, 10, 11, 12, rh10, or hu37 or a variant thereof.

78. The method of claim 77, wherein the capsid is from an AAV capsid of serotype 8 or 9 or a variant thereof.

79. The method of claim 78, wherein the capsid is from an AAV capsid of serotype rh10 or hu37 or a variant thereof.
